(19) United States
(12) Patent Application Publication
(10) Pub. No.: US 2018/0036225 A1
(43) Pub. Date: Feb. 8, 2018

(54) Compositions And Methods Comprising Yeast Organisms And Lipid Extracts Thereof

(71) Applicant: R.F. TECHNOLOGY CONSULTANTS, INC., Hollywood, FL (US)

(72) Inventors: Ramon F. Figueroa, Hollywood, FL (US); Maria Corena McLeod, Jacksonville, FL (US); Zvi G. Loewy, Fair Lawn, NJ (US)

(21) Appl. No.: 15/143,104

(22) Filed: Apr. 29, 2016

Related U.S. Application Data
(63) Continuation of application No. 15/137,820, filed on Apr. 25, 2016.
Application No. 15/137,820 is a continuation of application No. PCT/US2014/062464, filed on Oct. 27, 2014.
(60) Provisional application No. 62/154,344, filed on Apr. 29, 2015.
(60) Provisional application No. 61/895,490, filed on Oct. 25, 2013.

(51) Int. Cl. A61K 8/99; A61K 36/062; A23L 2/52; A61K 8/14

(52) U.S. Cl. CPC A61K 8/99; A61K 8/14; A61K 36/062; A23L 2/52; A61Q 19/08

(57) ABSTRACT

Disclosed herein are methods and compositions comprising whole yeast organisms and/or lipid yeast extract. Such compositions may be cosmetic, medical, biotechnological or agricultural compositions, comprising one or more cosmetic, medical, biotechnological or agricultural ingredients. The invention comprises methods of making such compositions. The yeast components of the compositions may be derived from yeast cultures which comprise at least 0.1% oil by dry weight.

### RELATED APPLICATIONS

This application claims the priority of and benefit of filing date of U.S. Provisional Patent Application No. 62/154,344, filed Apr. 29, 2016, and is a continuation of U.S. patent application Ser. No. 15/137,820, filed Apr. 25, 2016, which is a continuation of PCT/US2014/062464, which claims the priority of U.S. Provisional Patent Application No. 61/895,490, filed Oct. 25, 2013, each of which is incorporated herein in its entirety.

### TECHNICAL AREA

Disclosed herein are methods and compositions comprising yeast and yeast extracts useful in medical, biotechnical, agricultural and cosmetic compositions and methods using such compositions.

### BACKGROUND

The architectural framework of animal cells (cell or plasma membrane) represents a physical and biologically active membrane that separates the intracellular and extracellular environments of the cell. In a manner where structure is subservient to cellular function, the framework integrity of cell membranes is essential to cellular homeostasis by providing architectural support, intracellular and extracellular protection, osmotic regulation, select permeability of ions and organic nutrients, and ultimately optimal processes involved in cellular function.

The cell membrane framework consists of phospholipid bilayers which are embedded with proteins and other biological components within the framework to carry out functions such as ion conductivity and cellular signaling.

The cell membrane is selectively permeable, controlling or regulating the traffic flow of what is allowed to permeate in and out of the cell in either a passive or active manner. These include a number of transport mechanisms including osmosis and diffusion, transmembrane protein channels and transporters, endocytosis, and exocytosis to maintain a homeostatic cellular health and function.

Skin and mucosal cells for example, rely on cell membrane integrity to maintain homeostasis as they proliferate up to the outer layer of skin—the stratum corneum. Restoring, protecting and repairing the cell membrane against aging factors (intrinsic or extrinsic) would be a desired skin therapy benefit. What is needed are compositions and methods comprising isolated cellular membranes to provide dynamic delivery of lipids and proteins found in cellular membranes to cellular surfaces such as skin and mucosal surfaces.

### SUMMARY

The present disclosure comprises methods and compositions comprising yeast and yeast extracts useful in medical, biotechnical, agricultural and cosmetic compositions and methods of making and using such compositions. Disclosed herein are compositions and methods comprising yeast or lipid yeast extract, or a combination of both yeast and lipid yeast extract as disclosed herein, for example, yeasts may serve as a source of lipids for cosmetic and medical compositions and methods. Disclosed herein are methods and compositions comprising yeast disclosed herein, and compounds or compositions isolated, such as extracted, from such yeast, such as lipids, for methods and compositions, including, but not limited to, personal care compositions, food and nutritional compositions, pharmaceutical compositions, incorporation of compositions disclosed herein into medical devices, and methods of biotechnology and agriculture. Compositions and methods disclosed herein may provide dynamic delivery of yeast lipids, constituted liposomes made form yeast lipids, yeast proteins found in cellular surfaces and other products derived from yeasts to cellular surfaces such as skin and mucosal surfaces in medical and cosmetic treatments or applied to plants as biotechnological or agricultural remedies.

In an aspect, D. hansenii or other oleaginous yeast disclosed herein may be used in medical, biotechnical, agricultural and cosmetic compositions and methods. The yeast may be provided in the composition in a substantially whole cell form, in that the yeast cell is not lysed, but is provided relatively intact, or the entire cell may be lysed and all of the components of the yeast cell are provided. Lysed cells' components may be homogenized.

In an aspect, compositions and methods disclosed comprise a lipid yeast extract. Compositions may comprise a lipid yeast extract, for example, as liposome carriers made from the yeasts or compounds isolated from yeasts disclosed herein or liposome carriers may comprise phospholipids derived from yeast, such as D. hansenii. Compositions and methods disclosed herein may comprise extracts of other components of a yeast, such as D. hansenii. For example, extracts comprising proteins, carbohydrates, glycoproteins, nucleic acids, lipoproteins, organelles, cellular inclusions, or other yeast cell components or combinations thereof may be extracted (e.g. purified) from a yeast cellular lysate. Such extract compositions may be combined with lipid yeast extracts or extract compositions may be used individually.

In an aspect, compositions and methods disclosed comprise a lipid yeast extract. Compositions may comprise lipid yeast extract, for example, formulated as liposome carriers made from the yeasts or compounds isolated from yeasts disclosed herein or liposome carriers of medical or cosmetic actives, wherein the lipid yeast extract or liposomes made therefrom are derived from D. hansenii or more than one species of yeast. In an aspect, compositions and methods disclosed comprise a lipid yeast extract. Compositions may comprise lipid yeast extract, for example, as liposome carriers made from the yeasts or compounds isolated from yeasts disclosed herein, serving as integrated liposome carriers of medical or cosmetic actives derived from yeast, such as D. hansenii. Liposomes disclosed herein may be formed using known liposomal conventional techniques for producing liposomes such as sonication supercritical fluid technology, supercritical anti-solvent, supercritical reverse phase separation, and dual asymmetric centrifuging and other techniques for producing liposomes. Compositions may comprise a lipid yeast extract, for example, glycerophospholipid compounds of yeasts or phospholipid isolated from yeasts disclosed herein such as phospholipids derived from yeast, such as D. hansenii.

Compositions may comprise a specific lipid yeast extract, for example, free fatty acid, ceramide, sphingolipid, eicosinoid lipid, made from the yeasts or compounds isolated from yeasts disclosed herein, which may comprise such specific lipids derived from yeast, such as D. hansenii. Compositions may comprise lipid yeast extract, for example, sterol from the yeasts or isolated from yeast disclosed herein, such as ergosterol derived from yeast such as D. hansenii. Compositions may comprise a lipid yeast extract which also comprises non-lipid compound, for example, protein, terpene, polyphenol, carbohydrate, nucleic acid compounds made from the yeasts or isolated from yeasts disclosed herein such as derived from yeast, such as D. hansenii. In an aspect, compositions and methods disclosed comprise a combination of yeast cells and a lipid yeast extract, yeast cells and one or more extracts of yeast components other than lipids, or yeast cells, a lipid yeast extract and one or more extracts of yeast components other than lipids.

Methods and compositions may comprise effective treatments for lipid replenishment, such as replenishment of skin lipids for animal skin and/or hair. Compositions disclosed herein may be used for topical administration and provide enhanced transdermal penetration and delivery. Compositions of yeast and its extracts may be used for environmental remediation and for agricultural treatments of plants and/or soil. Active delivery vehicles such as liposomes, for example, comprising a lipid yeast extract from D. hansenii, can be used as vesicle carriers of medicaments or actives via administration by topical routes, transdermal patch, oral routes of administration including liquids, tablets capsules, or injectable compositions including, but not limited to, intradermnnal, subcutaneous, intramuscular, intravenous, intraosseous, intraperitoneal, intrathecal, epidural, intracardiac, intraarticular, intracavernous, or intravitreal. Liposomes made with the lipids of yeast disclosed herein can be produced by those of skill in the art using methods of producing microspheres or liposomes, for example by sonification. Formulations comprising emulsions that produce lamellar structures, such as liquid crystals, can be used in methods and compositions disclosed herein. Yeast extract liposomes may also be formed during sonication and disruption of the cell bodies of the disclosed yeast organisms, and liposomes disclosed herein may comprise such naturally forming liposomes.

In an aspect, disclosed are cosmetic or medical, biotechnical or agricultural compositions comprising at least 0.1% w/w whole yeast organisms yeast or lipid yeast extract, or a combination of both yeast and lipid yeast extract, and optionally, at least one other cosmetic or medical, biotechnical or agricultural ingredient. For example, a composition may comprise at least 10% whole yeast by dry weight, comprise at least 20% whole yeast by dry weight, at least 30% whole yeast by dry weight, at least 40% whole yeast by dry weight, at least 50% whole yeast by dry weight, at least 60% whole yeast by dry weight, at least 70% whole yeast by dry weight, at least 80% whole yeast by dry weight, at least 90% whole yeast by dry weight, or 100% whole yeast by dry weight. In an aspect, a composition may comprise at least 1% w/w yeast, which as used herein means the entire body of the yeast organism with its internal and external components, whether the body is intact (non-lysed yeast body) or not (lysed yeast body). In other aspects, a composition may comprise at least 10% w/w yeast. In an aspect, a composition may comprise at least 25% w/w yeast. In an aspect, a composition may comprise at least 50% w/w yeast. In an aspect, a cosmetic or medical composition disclosed is free of lipid other than lipid entrapped inside the yeast body. As used herein, “yeast” refers to the entire yeast organism, wherein the yeast organism is intact, as in not lysed, or all the components of an entire yeast organism are provided, such as from a whole intact yeast or from a lysed yeast organism. As is understood, “a yeast organism” refers to more than a single organism as multiple organisms are intended to be used in the compositions and methods disclosed herein. Where appropriate, two or more distinct species of yeast organisms, such as D. hansenii and Yarrowia lioplytica may be used in compositions and methods disclosed herein. In an aspect, a cosmetic, medical biotechnological or agricultural composition disclosed is free of lipid other than lipid entrapped inside the yeast body

In an aspect, disclosed herein are compositions comprising at least 0.1% w/w lipid yeast extract, and optionally, at least one other cosmetic or medical ingredient, in which the lipid yeast extract is derived from yeast disclosed herein. In an aspect, a yeast extract composition comprises 100% w/w lipid yeast extract by dry weight. In an aspect, a yeast extract composition comprises 10-90% w/w lipid yeast extract by dry weight. In an aspect, a composition comprises 25-80% w/w lipid yeast extract by dry weight. In an aspect, a composition comprises 35-70% w/w lipid yeast extract by dry weight. In an aspect, a composition comprises 45-60% w/w lipid yeast extract by dry weight.

In an aspect, disclosed herein are compositions comprising at least 0.1% w/w yeast and lipid yeast extract, and optionally, at least one other cosmetic, medical, biotechnical, or agricultural ingredient, in which the lipid yeast extract is derived from yeast disclosed herein. In an aspect, a yeast and lipid yeast extract composition comprises 10-90% w/w yeast and lipid yeast extract by dry weight. In an aspect, a yeast and lipid yeast extract composition comprises 25-80% w/w yeast and lipid yeast extract by dry weight. In an aspect, a yeast and lipid yeast extract composition comprises 35-70% w/w yeast and lipid yeast extract by dry weight. In an aspect, a yeast and lipid yeast extract composition comprises 45-60% w/w yeast and lipid yeast extract by dry weight. In such combinations, the yeast may be from 0.1%-99.9% of the combination of yeast and lipid yeast extract, and the lipid yeast extract may be from 99.9% to 0.1% of the combination of yeast and lipid yeast extract.

In an aspect, a yeast lipid extract composition may comprise a mixture of lipids extracted from at least two distinct species of yeast. In an aspect, a composition comprises a mixture of at least two distinct species of yeast, wherein the yeast are provided as whole yeast or lysed whole yeast comprising all the components of the whole yeast. In an aspect, at least two of the distinct species of yeast have been separately cultured. In an aspect, each distinct species has a lipid profile that is different from the other yeast species used in a composition. In an aspect, a composition comprises yeast comprising a mixture of at least two different yeasts, each yeast having a lipid profile different from the other yeasts. As used herein. “yeast” means one or more individual organisms and may comprise a plurality of yeast organisms.

In an aspect, disclosed herein is a method of making a cosmetic, medical, biotechnological or agricultural composition comprising combining yeast with optionally, at least one other cosmetic, medical, biotechnological or agricultural ingredient, to form a cosmetic, medical, biotechnological or agricultural composition. In an aspect, a method may comprise a method of making a cosmetic, medical, biotechnological or agricultural composition comprising combining a yeast lipid extract, extracted from yeast disclosed herein, with at least one other cosmetic, medical, biotechnological, or agricultural ingredient to form a cosmetic, medical, biotechnological or agricultural composition. In an aspect, a method comprises drying the yeast or extracted yeast lipids prior to combining the yeast or extracted yeast lipids, or a combination of yeast and extracted yeast lipids, with at least one other cosmetic, medical, biotechnological or agricultural ingredient.

In an aspect, disclosed herein is a method of making a cosmetic or medical composition comprising combining a compositions comprising yeast and a lipid yeast extract with optionally, at least one other (natural, synthetic, or biotechnological) cosmetic or medical ingredient, to form a cosmetic or medical composition. In an aspect, a method comprises drying the yeast or extracted yeast lipids prior to combining the yeast or extracted yeast lipids, or a combination of yeast and extracted yeast lipids, with at least one other cosmetic, medical, biotechnological or agricultural ingredient. In an aspect, a method disclosed may comprise a method of making a cosmetic, medical, biotechnological or agricultural composition comprising combining a lipid yeast extract with at least one other cosmetic, medical, biotechnological or agricultural ingredient to form a cosmetic, medical, biotechnological or agricultural composition. In an aspect, a method may comprise a method of using a yeast and/or lipid yeast extract composition for cosmetic purposes, such as to prevent or retard skin degradation and dehydration resulting from intrinsic or extrinsic induced factors, such as dehydration. In an aspect, a method may comprise a method of using a yeast and/or lipid yeast extract composition for cosmetic purposes, such as to soften and impart pliability to skin.

In an aspect, a method comprises contacting the external surface of an animal, for example, human skin, with a yeast or lipid yeast extract, or a combination of both yeast and lipid yeast extract composition. For example a composition may comprise intact yeast cells and at least 5% w/w lipid yeast extract by dry weight. For example, the lipid yeast extract may comprise from about 5% w/w lipid yeast extract by dry weight, from about 10% w/w lipid yeast extract by dry weight, 15% w/w lipid yeast extract by dry weight, 20% w/w lipid yeast extract by dry weight, 25% w/w lipid yeast extract by dry weight, 30% w/w lipid yeast extract by dry weight, 35% w/w lipid yeast extract by dry weight, 40% w/w lipid yeast extract by dry weight, 45% w/w lipid yeast extract by dry weight, 50% w/w lipid yeast extract by dry weight, 55% w/w lipid yeast extract by dry weight, 60% w/w lipid yeast extract by dry weight, 65% w/w lipid yeast extract by dry weight, 70% w/w lipid yeast extract by dry weight, 75% w/w lipid yeast extract by dry weight, 80% w/w lipid yeast extract by dry weight, 85% w/w lipid yeast extract by dry weight, 90% w/w lipid yeast extract by dry weight, or 95% w/w lipid yeast extract by dry weight. In an aspect, a method comprises retaining the composition in contact with the external surface, for example, skin, for a predetermined time period such as, for example, 30 minutes, 1 hour, or longer. In an aspect, a yeast and/or lipid yeast extract composition is retained in contact with an external surface, for example, skin for at least 3 hours. In an aspect, a method of using a yeast and/or lipid yeast extract composition further comprises maintaining the composition in contact with an external surface, for example, skin, for a period of time sufficient to release at least 50% w/w of the oil from intact yeast cells, which may occur, for example, by mechanical or non-mechanical (enzymatic) cell disruption leading to degradation of the yeast cell membrane to release oil.

In disclosed methods of using a yeast composition to soften and impart pliability to external surface of animals, such as skin and/or hair, a composition may comprise yeast cells containing at least 1-15% oil by dry weight. In an aspect, a composition may comprise yeast cells containing at least 35% oil by dry weight. In an aspect, a composition may comprise yeast cells containing at least 45% oil by dry weight. In an aspect, a composition may comprise yeast cells containing 15-90% oil by dry weight. In an aspect, a composition may comprise yeast cells containing 25-80% oil by dry weight. In an aspect, a composition may comprise yeast cells containing 35-70% oil by dry weight.

In an aspect, a composition may comprise yeast cells containing 45-60% oil by dry weight. In a cosmetic, medical, biotechnological or agricultural composition and/or method disclosed herein, a yeast cell may be one or more known yeasts including endophytic yeast isolate or endosymbiont. In an aspect, the yeast is an extremophile. In an aspect the yeast is Candida apicola, Candida etchellsii, Candida famata, Candida glabrata, Candida guilliermondii, Candida lactis-condens, Candida magnolia, Candida parapsilosis, Candida tropicalis, Candida versatilis, Citeromyces matritensis, Debaryomyces hansenii, Hanseniaspora guilliermondii, Hyphopichia burtonii, Issatchenkia orientalis, Kluyveromyces thermotolerans, Pichia angusta, Pichia anomala, Pichia farinose, Pichia guilliermondii, Pichia membranaefaciens, Pichia ohmeri, Schizosaccharomyces octosporus, Schizosaccharomyces pombe, Torulaspora delbrueckii, Zygosaccharomyces Bailii, Zygosaccharomyces bisporus, Yarrowia lipolytica, Zygosaccharomyces microellipsoides, and Zygosaccharomyces roux.

In a composition and/or method disclosed herein, a cosmetic or medical ingredient may be one or more of absorbents, abrasives, anticaking agents, antifoaming agents, antimicrobial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances, flavor oils, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, soaps, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, vitamin derivatives, and natural extracts. In at least one embodiment, the other cosmetic or medical ingredient comprises a soap. In some cases, the soap comprises a saponified oil derived from yeast.

In an aspect, a method may comprise using a yeast, and/or yeast extract composition for bioremediation purposes, such as to elimination noxious by-products, such as those by-products of the coffee industry. In an aspect, a method comprises contacting the noxious byproducts with a yeast and/or a yeast extract, or a combination of both yeast and yeast extract composition. For example, a composition comprises intact yeast cells and at least 10% w/w yeast extract by dry weight. In an aspect, a method comprises retaining the composition in contact with the noxious by-products for a predetermined time period such as, for example, one day, one week or longer. In an aspect, the yeast, and/or yeast extract composition is retained in contact with the noxious by-products for at least one month.

In an aspect, a method may comprise a method of using a yeast, and/or yeast extract composition for the treatment of agricultural diseases, such as the elimination of fungus of the Emileia vastatrix sp, Fusarium sp, Phytophthora sp, and Rhizoctonia sp. In an aspect, a method comprises contacting the external parts of the plant with a yeast, and/or a yeast extract, comprising intact yeast cells and at least 10% w/w yeast extract by dry weight. In an aspect, a method comprises retaining the composition in contact with the external part of the plant for a predetermined time period such as, for example, one day, one week or longer. In an aspect, a yeast, and/or yeast extract composition is retained in contact with the external parts of the plant for at least one month. Compositions or methods disclosed herein can be combined together and are encompassed with the scope of the present disclosure.

### DESCRIPTION OF FIGURES

FIG. 1A AND FIG. 1B show liposome vesicles with a unilamellar and multi-lamellar lipid bilayer in the sample. These range in size from 60-350 nm in diameter with bilayer widths from 7-9 nm. Magnification is 52,000×. Scale Bar: 200 nm.

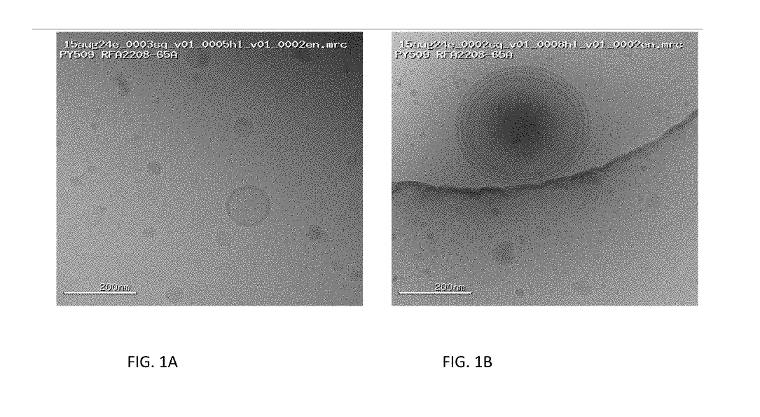

FIG. 2 is a graphic representation of cell numbers after pre-treatment of human dermal fibroblasts with different experimental conditions (before dehydration). Note the relative increase of cell viability as compared to Air control, except for 3C at 10%, which was cytotoxic.

### DETAILED DESCRIPTION

The present disclosure comprises methods and compositions comprising yeast and yeast extracts useful in medical, biotechnical, agricultural and cosmetic compositions and methods using such compositions.

Disclosed herein are yeast extracts comprising a components of cellular membranes, including, but not limited to liposomes derived from yeast cellular membranes. Methods of providing compositions comprising yeast extracts, such as liposomes or lipid components extracted from yeast disclosed herein comprise repair, protection from microbial infection, and maintenance of skin and mucosal surfaces. Cellular membranes comprise mostly phospholipids, proteins, glycoproteins and depending on the source of the cellular membrane (organism), a sterol, for example ergosterol or cholesterol.

The cell membrane of all cells is subject to damage and degradation. For example, under stress, cell membrane components may undergo oxidation. Damage to cellular membrane compromises cellular functions and can lead to DNA damage, compromising replication and accelerating cellular death (apoptosis). An example of damage could be oxidation as in reactive oxygen species (ROS) and their interactions within cell membrane components which can directly or indirectly compromising cellular function. Lipid oxidation or lipid peroxidation may contribute to loss of cellular function by inactivating enzymes and water soluble proteins to inhibit optimal architectural support, compromising normal intracellular and extracellular protection, osmotic regulation, and permeability of ions and organic nutrients. Furthermore, lipid peroxidation within cell membrane phospholipids has been found abundant at sites of inflammation, For example, lipid peroxidation plays an active role in the modulation of the immune response. Studies have shown effects of oxidation-specific-epitopes (OSE's) and their effect on the innate immune response as a result of phospholipid peroxidation with correlation to disease states including for example arteriosclerosis and age related macular degeneration. See for example, http://www.ncbi.nlm.nih.gov/pmc/articles/PMC3790971/.

Phospholipids are major components of cell membrane structure due to their amphiphilicity. Their hydrophobic tail and hydrophilic head provide phospholipids the ability to form lipid bilayers. These bilayers are made up of lipid classes of phospholipids, sphingolipids, and sterols such as phophatidylcholine, sphingomyelin, and cholesterol, respectively. Other phospholipid components include phophophatidylserine, phosphatidylehtanolamine, phosphatidylinositol, and phosphtadildylglycerol. The lipid bilayer creates a practically impermeable barrier to the interior and exterior of the cell.

Certain phospholipid classes can be predominant depending on cell type. For example, phosphatidylserine is important to cellular signaling in brain or neuron cells, promoting cell signaling synapses for optimal cognitive function. Phospholipids also are building blocks for cellular organelle membranes such as mitochondria. For example, cardiolipin is a phosphatidylglycerol compound important to the inner mitochondrial membrane and is essential to enzymes involved in mitochondrial energy metabolism.

In skin, lipids play many roles. For example, lipids play a role in the formation and maintenance of both the permeability and antimicrobial lamellar barriers of skin. This hydrophobic lamellar extracellular lipid matrix in the stratum corneum is composed of lipids such as phospholipids, sphingolipids, ceramides and cholesterol, contributing to the barrier, cohesion, antimicrobial and other metabolic functions within skin. Maintenance of skin hydration is greatly dependent on the extracellular lipids by reducing trans-epidermal moisture loss. Topical products attempt to use hydrophobic emollients in the form of hydrocarbons, esters, silicones, and natural oils to ameliorate skin dryness and reduce trans-epidermal moisture loss. Phospholipids derived from soy, egg or other sources have also been used to help restore the natural lamellar extracellular bilayers. For example, a company named Lipoid has recently launched SLM 2038 Skin Lipid Matrix, a multilamellar cream produced from non-GMO sunflower phospholipids and is applicable in skin care products to protect the skin and support its natural barrier function by replacing missing barrier-lipids. Such plant-derived phospholipids may provide some replacement for barrier lipids, but what is needed is a source of phospholipids that provides an greater array of “biologic similars” or phospholipids that provides a greater “biologic fit” as an approach to restoration, assimilation, and repair of the skin cell membrane and extracellular spaces between skin cells to relieve dryness and restore, maintain and retain skin moisture of the skin.

Though not wishing to be bound by any particular theory, it is believed that yeast cells have greater homology to animal cells than plant cells. For example, in animal cell membrane the variation in head groups, aliphatic chains and alcohols leads to the existence of a wide variety of phospholipid isomers and enantiomers creating a unique and personalized architectural framework. Various plant based commercial phospholipids, such as soybean phosphatidylcholine, egg phosphatidylcholine, or synthetic phosphatidylcholine are commonly used to attempt to deliver, transport, and assimilate into cell membrane. However, the current sources of phospholipids such as soy have not been demonstrated to possess the diversity of geometric and positional isomers of the unsaturated fatty acids found in diversified animal cell membranes. In molecular species selectivity of the lipid metabolism, phospholipid composition and synthesis are similar in yeast and mammalian cells. Further, many of the processes used for extraction or treatment during or after extraction (i.e. hydrogenation) result in phospholipid physical properties that differ from the naturally occurring cell membrane isomers present. Presence of these unnatural isomers has led to extensive investigation of their nutritional value and biological properties. For example, phosphatidylserine molecule from soy lecithin contains mainly polyunsaturated acids, while the phosphatidylserine molecule from brain cortex contains mainly saturated and monounsaturated fatty acids and long-chain polyunsaturated fatty acids. Disclosed herein are compositions providing lipids that are more homologous to animal cells that provide a broader spectrum or array of biocompatible or “biologically correct” phospholipids, and which can provide a correct biologically membrane fit approach to biocompatibility, assimilation, delivery and repair of cell membrane to restore optimal cellular function.

Compositions of the present disclosure provide more homologous phospholipids to mammal, such as human. Lipids produced by yeast have greater diversity than plant lipids, and thus compositions disclosed herein provide one or more lipids that are biologically more similar to skin and can lead to greater restoration, assimilation, and repair of skin cell membrane and the extracellular spaces between skin cells to relieve dryness and restore, maintain and retain skin moisture of the skin.

There are many food sources rich in phospholipids. However, many of the rich dietary sources of phospholipids are also high in cholesterol and fat. For example, egg yolk, meat and internal organs are good sources of phospholipids but can be high in cholesterol. Grains, fruits and vegetables are low in cholesterol but also are relatively low in their phospholipid concentration. Therefore, with the concerns for fat and cholesterol, modern diets provide minimal phospholipid supplementation. It is estimated that today's diet contains significantly less phospholipid levels over the last century.

Oleaginous yeast are capable of lipid production. Yeast such as Cryptococcus podzolicus, Trichosporon porosum, Pichia segobiensis, and Candida freyschussii have been reported as beneficial for microbial production processes for industrial lipid production. The lipids produced by these yeast consist of fatty acids and triglycerides, but not phospholipids.

Extremophile oleaginous yeast and bacteria have been described as capable of producing a diversity of phospholipids. These include marine and wetland (aquaphile extremes) such as halophiles and acidophiles among other extremophiles. For example, an acidophile reported from a Sphagnum peat bog described Bacillus acidiola produces lipids such as diphosphatidylglycerol, phosphatidylglycerol and phosphotidylethanolamine. Another polyextremophile, an osmotolerant, halotolerant, xerotolerant, cryotolerant, acidotoloerant, oleaginous yeast described as Debaryomyces hansenii has been reported to produce phospholipids such as phosphatidylcholine, phosphatidylinositol, phosphoethanolamine, phosphatidylserine, phosphatidylglycerol, and cardiolipin. See PCT Application Serial No. PCT/US2014/062464, which is incorporated herein in its entirety.

There are reports concluding the diversity of intracellular lipids and how lipid metabolism is dramatically perturbed in numerous metabolic diseases stemming from genetic and/or dietary/nutritional and life style, for example type 2 diabetes, cardiovascular disease, autoimmune disorders, rheumatoid arthritis, neurodegenerative diseases, kidney and liver disorders. There have also been studies on the effect of stress, for example brain lipid profiles and stress. There have also been studies suggesting endurance during exercise can be enhanced with phospholipid supplementation.

Therefore, there is a need for selective nutritional supplementation of essential phospholipids. Compositions disclosure herein comprise a source of phospholipids that provides an array of “biological equivalents of biosimilars” or phospholipids which provides a greater “biologic fit” for humans, for example, to replenishment and improvement in an individual's lipid profile to promote health and enhanced performance. Methods disclosed herein comprise a total systems approach including: obtaining an individual's qualitative and quantitative lipid profiles in real time to define the nutritional or metabolic needs, meaning the lack of one or more phospholipids, of an individual to achieve optimal health and performance. In addition, methods comprise identifying biochemical and genomic pathways to be integrated in a process that allows for the diagnostic evaluation of an individual.

Attempts to utilize commercially available phospholipids to restore, replenish, and repair cellular membranes as well as using phospholipids as transport delivery vehicles exist. Commercial sources of phospholipids include bovine (egg) and plant (soybean). Lecithin as a source of commercial phospholipids represents the vast majority of non-synthetic commercial phospholipids (>90%). Lecithin phospholipids represent greater than 80% of the world supply for industrial application. Lecithin is mostly extracted from natural sources such as soy beans and egg, and milk, marine sources, rapeseed, cottonseed, and sunflower are also known sources of lecithin. The process of separation and isolation of phospholipids from lecithin consist of using solvents such as hexane, ethanol, acetone, petroleum ether, or benzene. Phosphatidylcholine for example is the most abundant phospholipid derived from egg (approx. 70%) and soy (approx. 35%).

Almost 90% of commercial lecithin phospholipids is produced from soy. Almost all soy produced in the US is genetically modified (GMO) soy. Soy is an important food in the global supply chain. It is projected that feeding the growing global population by 2050 will be one of the biggest challenges confronting our planet. What is needed is a non-food, non GMO source of commercial phospholipids. Eggs for somewhat similar reasons provide sound reasons to find alternative source of phospholipids.

Due to their amphililic nature, lecithin phospholipids have emulsifying and solubilizing properties and extensively used in cosmetics, foods, and other product segments. Lecithin and phospholipids are supplied in various grades. For example, hydrogenated lecithin, a major emulsifier containing phospholipids, is made by reacting hydrogen with lecithin resulting in a very stable waxy material. Lysophosphatidylcholine is produced by the partial hydrolysis of phosphatidylcholines, which removes one of the fatty acid groups. Among the major drawbacks to soy phospholipids are color, odor, and residual solvents. Major global suppliers of soy phospholipids include VAV Science, Lipoid/American Lecithin, Cargill, Lecico, ADM, and others.

Optimal cellular membrane integrity promotes healthier cellular function. Reduction in select phospholipid and increase in phospholipase has been shown in inflamed skin. For example, a decrease of major cytoplasmic membrane phospholipids (phosphatidylcholine and phosphatidylethanolamine) content was established in mucosal epithelial cells under colon inflammation pathology.

Liposomes

Due to their amphilicity of molecules which orients a hydrophilic head of lipid molecules to be located at the lipid water interface, phospholipids have a self-assembly spherical bilayer-forming capability of vesicles or liposomes which surround an aqueous internal compartment. Liposomes model biological membranes of both eukaryotes and prokaryotes. Liposomes are capable of shielding or protecting sensitive compounds from oxidation, pH, and enzymatic or chemical changes with components outside the protectant vesicle.

Most available liposomes are formed from either synthetic, egg or soy derived phospholipids. Plant cells such as soy are not as homologous with mammalian cells as yeast cells. Disclosed herein are compositions comprising homologous phospholipids that provide an array of “biologically similars” or phospholipids that provide a “correct biologically membrane fit” as an approach to liposomal delivery for the restoration, assimilation, and repair of cell membrane to restore optimal cellular function. Liposomes disclosed herein are made from yeast cells, particularly, D. hansenii yeast, and may comprise yeast lipid extract compositions disclosed herein. Liposomes disclosed herein may be made by methods known to those of skill in the art (e.g., Bangham, sonication, reverse phase), and liposomes may be formed during the process of disrupting yeast cells, such as after sonification of yeast cells, as disclosed in Example 1.

Though not wishing to be bound by any particular theory, it is believed that yeast phospholipids disclosed herein have enhanced activity, for example, assimilation with eukaryotic (e.g., human or animal) cell membranes when compared to soy or egg phospholipids. There is better assimilation and delivery with the phospholipids of yeast. Liposomes made from lipids extracted from microorganisms disclosed herein, such as D. hansenii, show better assimilation with human or animal skin for better delivery of components of the liposomes.

Liposomes can be administered in usual routes of administration, including but not limited to, topically, ocularly, pulmonary, nasally, orally, intramuscularly, subcutaneously, or intravenously, and are widely used in cosmetics and medical targeted release applications including for active drug delivery, medical diagnostic, photodynamic therapy, anti-cancer and gene therapy, vaccination among others medical applications. In the food industry for example liposomes have been used to deliver terpenes to deliver food flavors and antimicrobials.

Liposomes are also biocompatible and biodegradable delivery systems demonstrating a versatile ability to transport hydrophilic, hydrophobic, and amphipathic therapeutic compounds which display enhanced permeation and assimilation to cell membrane structure. Their limitations are based on design and finding biologically correct phospholipid components to better assimilate in cell membrane. Disclosed herein are compositions comprising liposomes made from yeast to provide a more homologous-to-animal source of phospholipids that provides an array of “biologically correct” phospholipids to form biosimilar delivery systems in the form of liposomes for improved delivery.

Liposomes are usually created by a multi-step process of sources amphililic components from diverse sources (i.e. soy, egg) with specialized equipment. Disclosed herein are methods comprising an extraction process capable of creating liposomes in situ during extraction. Furthermore, liposomes are usually created via a non-integrated approach, being that phospholipids from one source (i.e. egg or soy), whereas the active material to be entrapped or encapsulated in a liposomes comes from another source. Disclosed herein are methods and compositions comprising an integrated approach to liposomes where the lipids used to form liposomes are derived from a source (i.e. yeast) and the active being encapsulated or entrapped within such liposome would be derived from the same source (i.e. yeast peptide), thus creating a fully integrated delivery system.

Disclosed compositions provide a diverse array of biologically correct phospholipids that may be used to customized specialized phospholipid liposomes which have significant advantages and applications. For example, the nasal cavity is covered by a thin mucosa which is well vascularized and receptive to enhanced permeability provided by disclosed liposomes. An active agent, such as a drug molecule, can be transferred quickly across the single epithelial cell layer directly to the systemic blood circulation without first-pass hepatic and intestinal metabolism. Greater permeability and assimilation offered by biologically correct phospholipid liposomes effectively deliver local and systemic treatments nasally. For example, liposomal nasal administration can be used to deliver relief of nasal dryness, or decongestant allergy treatments, or systemic treatments such as active agents for migraine headaches. Specialized phospholipid liposomes might demonstrate advantages for intranasal delivery of active compounds that can cross the blood-brain barrier for neurological disorders. Disclosed herein are compositions comprising liposomes made from yeast to provide more homologous-to-animal source of phospholipids that provides an array of “biologically correct” phospholipids to form biosimilar delivery systems in the form of liposomes for improved delivery.

Liposomes have been investigated for ophthalmic drug delivery since it offers advantages as a carrier system. They can treat dry eye conditions or enhance the permeation of poorly absorbed drug molecules by binding to the corneal surface and improving residence time. Liposomes can improve pharmacokinetic profile, enhance therapeutic effect, and reduce toxicity associated with higher dose. Disclosed herein are compositions comprising liposomes made from yeast to provide a more homologous-to-animal source of phospholipids that provides an array of “biologically correct” phospholipids to form biosimilar delivery systems in the form of liposomes for improved delivery.

Delivery of Proteins

The overall surfaces of membrane proteins are mosaics, with patches of hydrophobic amino acids where the proteins contact phospholipids in the membrane bilayer, and patches of hydrophilic amino acids on the surfaces that extend into the water-based cytoplasm. The proteins in the cell membrane typically help the cell interact with its environment. For example, cell membrane proteins carry out functions as diverse as transporting nutrients across the membrane, receiving chemical signals from outside the cell, translating chemical signals into intracellular action, and sometimes anchoring the cell in a particular location.

Embedded in cellular membrane with phospholipids, proteins help support the integrity of cellular structural framework, perform the role of transporter, and can also be messengers or conduits to cellular signaling. In addition, protein function can be performed as enzymes promoting thousands of chemical reactions responsible for a many cellular metabolic processes. Many proteins can move within the plasma membrane through a process called membrane diffusion. These membrane-bound proteins can travel within the membrane, creating a fluid-mosaic structure throughout the cell membrane. The portions of membrane proteins that extend beyond the lipid bilayer into the extracellular environment are also hydrophilic and are frequently modified by the addition of sugar molecules. And as antibodies proteins found in cell membrane, they can capture and bind foreign invaders such as viruses and bacteria to protect the cell. Hence, cellular proteins, which are made up of thousands of smaller amino acid units, play important role in cellular structure, function, regulation and protection.

Observations have been reported suggesting that many homologous proteins in bacteria, yeast, and humans have been conserved stringently in several phylogenetic lines. Disclosed herein are compositions comprising liposomes made from yeast to provide a more homologous source of phospholipid protein complex that can provides a broader spectrum or array of biocompatible or “biologically correct” nutrients to provide a correct biologically membrane fit approach to biocompatibility, assimilation, delivery, repair, transport and signaling of cell membrane to restore optimal cellular function.

Yeast have been reported to produce cytotoxic proteins or ‘killer toxins’ to protect against other yeast or as a way of gaining a competitive advantage for limited nutritional resources over other strains. These proteins bind to the cell wall in a receptor-mediated way and are subsequently translocated to the cell membrane. Disclosed herein are compositions comprising liposomes made from yeast to provide a more homologous-to-animal cell source of select and biocompatible cytotoxic proteins which can provide protection against other yeast, for example Candida albicans.

Candidiasis is a yeast infection that can attack skin and mucosal tissue of the mouth, throat, and genital epithelial cells. Decrease of major cytoplasmic membrane phospholipids (phosphatidylcholine and phosphatidylethanolamine) has been linked in compromised epithelial cells. Disclosed herein are compositions comprising liposomes made from yeast to provide a more homologous-to-animal cell source of phospholipid-killer toxin complex that can provides a broader spectrum or array of biocompatible or “biologically correct” phospholipids, along with select killer toxins from yeast to repair cell membrane to restore optimal cellular function. Methods of the present disclosure comprise treating a candidiasis condition or infection in a person, comprising, administering an effective amount of a lipid extract composition or a liposome composition disclosed herein to a person having a yeast infection, and ameliorating, modulating, lessening or treating the yeast infection.

Yeast toxins might provide select inhibitory effects on skin. Cytochalasin B for example, a fungal toxin has been shown to inhibit actin filaments and thus restoring elasticity to skin. Compositions disclosed herein may comprise one or more yeast toxins. The fungal toxin cytochalasin B reduces skin cell size. With aging, skin cells become rigid and grow larger. Cytochalasin B can restore elasticity to skin cells and may shrink skin cells Older skin cells shrunk 20-40% in response to the compound, whereas younger skin cells didn't shrink significantly.

Production and Delivery of Terpenes

Terpenes are derived biosynthetically from units of isoprene, a very diverse class of compounds found in a variety of plants. They are also present in microorganisms of fermentation such as yeast. Terpenes represent the building blocks for essential oils and resins, and are often used in food additives, perfumery and aromatherapy. Terpenes have been shown to also have medicinal properties, helping fight bacteria and fungus. Specifically, they have shown to possess antitumor, antibacterial, antifungal, and anti-parasitic activity. Terpenes are of interest for their high value use in food, cosmetic, pharmaceutical and biotechnological industries. Chemical synthesis of terpenes can be problematic because of their complex structure, and because plants produce very small amounts of these valuable chemicals, it is difficult, time consuming, and expensive to extract them directly from plants. Disclosed herein are compositions and methods that have been optimized to produce select terpenes, under a process of stress, for example, for industrial applications.

Terpenes have also been explored as skin penetration enhancers. Aside from increasing the solubility of drugs into skin lipids and lipid membranes, their mechanism for percutaneous permeation enhancement involves disruption of lipid-protein organization which leads to degradation of constituents that are responsible for maintenance of cellular structure and membrane barrier. Disclosed herein are compositions that provide a more homologous-to-animal cell source of phospholipid-terpene complexes that can provides a broader spectrum or array of biocompatible or “biologically correct” phospholipids, without or by minimizing cell membrane disruption for the maintenance of cellular function.

Of particular interest are antimicrobial terpene compounds such as 2-phenoxy ethanol and farnesol which are currently preservatives used in cosmetics and other industries. Of further particular interest are prenol lipids dilichols and ubiquinones.

Protein Amplification

Eukaryotic cells are compartmentalized into structures called organelles. Organelles within a cell differ one from another by both structure and function. The protein composition of organelles, i.e. mitochondria vs. golgi vs. endoplasmic reticulum, are unique. Hence, the proteomic complexity can be reduced by isolating an organelle, and analyzing the organelle's proteome, as opposed to analyzing the cell's proteome. Organelle proteomics is attractive as a method for analysis of cellular proteins. However, it is difficult to isolate organelles to homogeneity.

A method of the present disclosure comprises using liposomes disclosed herein to interact with organelle membranes, to tag the protein membrane, and facilitate the selective purification and enrichment of organelles. The potential reduction in complexity, coupled with the enrichment of organelles allows for protein amplification.

In an aspect, liposomes are made from lipids derived from extremophiles, including but not limited to Debaryomyces hansenii, which produce a broad array of unique lipids.

In an aspect, liposomes disclosed herein are attached to a solid support. Lysed tissues and cells in solution are contacted with the bound liposomes. The liposomes attach to protein membrane fractions, and capture unique membrane protein fractions.

In an aspect, liposomes disclosed herein are characterized, and specific liposomes are used in preparative systems for large scale membrane protein isolation. Specific liposomes can be attached to magnetic microparticles and used to enrich the protein membrane fractions in a continuous process. Proteins embedded in the membrane fractions isolated by the liposome capture technology can be analyzed by proteomic methodologies including 2D gel electrophoresis and mass spectrometry. Identification of proteins is facilitated by data base interrogation of the mass spectrometry sequences.

Lipo-Nutragenics

Phospholipids are key cellular macromolecules contributing to cell structure and function. The qualitative and quantitative composition of phospholipids varies from individual to individual. Often there is a nutritional need to supplement essential phospholipids. Disclosed is a total systems approach including: 1. obtaining an individual's genetic information, for example, through the use of gene chip technology; 2. obtaining an individual's qualitative and quantitative lipid profiles, for example by using lipid microarrays. This provides genetic and biochemical nutritional information detailing an individual's lipid needs. The nutritional needs can be ascertained from this genetic and lipid profile. Information, such as the lack of particular lipids, or an inadequate or overabundance of one or more lipids, of an individual can thus be defined using the process. Because of the unique attributes of microorganisms disclosed herein, for example, Debaryomyces hansenii, to produce diverse lipid profiles, compositions derived from such microorganisms can be used in methods of treatment to provide the essential phospholipids that are detected as missing using this genetic/biochemical process. A lipid yeast extract compositions disclosed herein can supply phospholipids in a personal medicine formulation. In an aspect, the biochemical and genomic testing is integrated in a process that allows for the diagnostic evaluation of an individual.

In an aspect, informatics by direct electronic connection can link the genetic/lipid profile determined from the subject and allow the subject or another to order essential phospholipids commercially. Informatics flow: Manage consumer care>molecular diagnostic profile>data analysis and need determination>order entry>delivery of phospholipids>measure performance—

In an aspect, novel essential phospholipids derived from extremophiles, such as Debaryomyces hansenii, are the source for the nutrient supply to the consumer.

Lipid Nutrigenics

Phospholipids are key cellular macromolecules contributing to cell structure and function. The qualitative and quantitative composition of phospholipids varies from individual to individual. Often there is a nutritional need to supplement essential phospholipids. The subject invention provides for a total systems approach including: 1. the ability to obtain an individual's complete genetic information through the use of gene chip technology; 2. obtain an individual's qualitative and quantitative lipid profiles (lipid microarrays) in real time by using specialized lipid micro-arrays. This provides genetic and biochemical nutritional information detailing an individual's lipid needs. The “nutritional needs” of an individual can thus be defined using the above process. Because of the unique attributes of extremophiles, such as Debaryomyces hansenii, to produce diverse lipid profiles, the extremophiles are a source of the essential phospholipids that are detected using the genetic/biochemical process disclosed. The microorganisms, such as Debaryomyces hansenii, can supply phospholipids in a personal format.

Disclosed herein are methods and compositions comprising lipids, such as yeasts comprising lipids, or extracts from yeasts, of which phospholipids are an example of lipids. Phospholipids are an important class of lipids in cell structure due to their amphiphilicity. Phospholipids are the major components of cell membranes. Their hydrophobic tail and hydrophilic head provide phospholipids to form lipid bilayers. These bilayers are made up of phospholipids, sphingolipids, and sterols such as phophatidylcholine, sphingomylin, and cholesterol respectively. Other phospholipid components of importance include phophophatidylserine, phosphatidylehtanolamine, and phosphtadildylglycerol. The lipid bilayer creates a practically impermeable barrier to the cells.

In skin, lipids play an essential role in the formation and maintenance of both the permeability and antimicrobial barriers. A hydrophobic extracellular lipid matrix in the stratum corneum is composed primarily of lipids, such as phospholipids, sphingolipids, and cholesterol contributing to the barrier, cohesion, antimicrobial and other metabolic effects.

Phospholipids such as phosphatidylcholine when combined with phospholipid surfactants such as phastidylethanolamine under high sheer have been shown to artificially produce spherical cell-like membrane vesicles such as liposomes. Liposome have been shown useful as carriers for enhance permeability and delivery of nutrients and pharmaceutical drugs. Liposomes have been commercially produced for multiple applications. Liposomes may be made methods known to those of skill in the art.

An example of a yeast useful in the methods and compositions disclosed herein is Debaryomyces hansenii, though the invention is not limited to only one species of yeast, and the references herein to a particular yeast is for clarity and not to be seen as limiting. Debaryomyces hansenii is an oleaginous yeast with roughly 70% w/w lipid content. Though not wishing to be bound by any particular theory, it is thought that major phospholipids in D. hansenii are phosphatidylcholine, followed by phosphatidyl inositol, phosphatidylethanolamine, phosphatidylserine, phophatidylglycerol and cardiolipin.

Yeast can be used to produce lipids economically, for example, for use in cosmetic or medical methods and compositions. A yeast disclosed herein for use in the invention is the lipid-producing yeast Debaryomyces hansenii. Disclosed herein are methods of culturing Debaryomyces hansenii as well as multiple other species of yeast to generate lipids for use in cosmetic or medical compositions. Any species of yeast that produces suitable oils and/or lipids can be used in accordance with the present disclosure, although yeast that produce high levels of suitable oils and/or lipids are effective for methods and compositions disclosed herein.

Considerations for selection of yeast for methods and compositions disclosed herein, in addition to production of suitable oils or lipids for compositions, include, but are not limited to (1) high lipid content as a percentage of cell weight; (2) ease of growth; (3) ease of propagation; (4) ease of biomass processing; (5) lipid profile and (6) lack of toxins. In an aspect, the yeast must be disrupted during the use of the cosmetic or medical composition (e.g., soaps containing whole yeast cells) in order to release the lipid components. Hence, in some compositions it is advantageous to comprise strains of yeast susceptible to disruption, such as when the yeast is to be used as whole yeast cells as an ingredient in the final cosmetic or medical composition.

In an aspect, wild-type or genetically engineered yeast comprise cells that are at least 10%, at least 15%, at least 20%, at least 25%, at least 30%, at least 35%, at least 40%, at least 45%, at least 50%, at least 55%, at least 60%, at least 65%, at least 70%, at least 75%, or at least 80% or more, oil by dry weight. Processing considerations can include, for example, the availability of effective means for lysing the yeast cells. In an aspect, not all types of lipids are desirable for use in cosmetics or medicine or as cosmetic or medical ingredients, as the lipids may have aesthetic issues, such as smelling bad, having poor stability or providing a poor tactile sensation.

Yeasts useful in accordance with the methods disclosed herein are found in various locations and environments throughout the world. As a consequence of their isolation from other species and their resulting evolutionary divergence, the particular growth medium for optimal growth and generation of whole yeast and/or yeasts for lipid yeast extract from any particular species of yeast may be determined by those of skill in the art who can readily find appropriate media by routine testing for growing yeast. In some cases, certain strains of yeasts may be unable to grow on a particular growth medium because of the presence of some inhibitory component or the absence of some essential nutritional requirement required by the particular strain of yeast. The fixed carbon source is a component of the medium for growing yeast. Suitable fixed carbon sources, include, for example, glucose, fructose, sucrose, galactose, xylose, mannose, rhamnose, arabinose, trehalose, N-acetylglucosamine, glycerol, floridoside, glucuronic acid, and/or acetate.

In a steady growth state, the yeast cells may accumulate oil but do not undergo cell division. In an aspect, the growth state is maintained by continuing to provide all components of the original growth media to the cells with the exception of a particular component of the media. Cultivating yeast cells by feeding all nutrients originally provided to the cells except for a particular component, such as through feeding the cells for an extended period of time, results in a higher percentage of lipid by dry cell weight. Yeast grown using conditions described herein or otherwise known in the art can comprise at least about 20% lipid by dry weight, and often comprise 35%, 45%, 55%, 65%, and even 75% or more lipid by dry weight. Percentage of dry cell weight as lipid in yeast lipid production can therefore be improved by holding cells in a heterotrophic growth state in which they consume carbon and accumulate oil but do not undergo cell division.

High protein biomass from yeast is another material for inclusion in cosmetic or medical compositions disclosed herein. A method of growing yeast may comprise growing yeast so that the yeast comprises a biomass that is at least 30% of its dry cell weight as protein. Growth conditions can be adjusted to increase the percentage weight of yeast cells that is protein. Such methods may be known to those of skill in the art or disclosed herein.

A bioreactor or fermenter may be used to culture yeast cells through the various phases of their physiological cycle. As an example, an inoculum of lipid-producing yeast cells is introduced into a medium; there is a lag period (lag phase) before the cells begin to divide and reproduce (propagate). Following the lag period, the propagation rate increases steadily and enters the log, or exponential, phase. The exponential phase is in turn followed by a slowing of propagation due to decreases in nutrients such as nitrogen, increases in toxic substances, and quorum sensing mechanisms. After this slowing, propagation stops, and the cells enter a stationary phase or steady growth state, depending on the particular environment provided to the cells. For obtaining protein rich biomass, a yeast culture is typically harvested during or shortly after the end of the exponential phase. For obtaining lipid rich biomass, a yeast culture is typically harvested well after the end of the exponential phase, which may be terminated early by allowing a key nutrient (other than carbon) to become depleted, forcing the cells to convert the carbon sources, present in excess, to lipid. Culture condition parameters can be manipulated to optimize total oil production, the combination of lipid species produced, and/or production of a specific lipid.

Bioreactors offer many advantages for use in growth and propagation methods. To produce biomass for use in cosmetics or medical compositions, yeast are preferably fermented in large quantities in liquid, such as in suspension cultures as an example. Bioreactors such as steel fermenters (5000 liter, 10,000 liter, 40,000 liter, and larger) can accommodate very large culture volumes. Bioreactors also typically allow for the control of culture conditions such as temperature, pH, oxygen tension, and carbon dioxide levels. For example, bioreactors are typically configurable, for example, using ports attached to tubing, to allow gaseous components, like oxygen or nitrogen, to be bubbled through a liquid culture.

Increased gas flow affects the turbidity of the culture as well. Turbulence can be achieved by placing a gas entry port below the level of the aqueous culture media so that gas entering the bioreactor bubbles to the surface of the culture. One or more gas exit ports allow gas to escape, thereby preventing pressure buildup in the bioreactor. Preferably a gas exit port leads to a “one-way” valve that prevents contaminating microorganisms from entering the bioreactor. The specific examples of bioreactors, culture conditions, and growth and propagation methods described herein can be combined in any suitable manner to improve efficiencies of microbial growth and lipid and/or protein production.

Yeast cultures generated according to methods disclosed herein yield yeast in fermentation media. To prepare the yeast for use as a cosmetic or medical composition, the yeast is concentrated, or harvested, from the fermentation medium. At the point of harvesting the yeast from the fermentation medium, the yeast comprises predominantly intact cells suspended in an aqueous culture medium. The present disclosure is not limited by the disclosed methods for concentrating yeast, as those of skill in the art are well aware of many methods to accomplish concentration of yeast. For example, to concentrate the yeast, a dewatering step may be performed. Dewatering or concentrating refers to the separation of the biomass from fermentation broth or other liquid medium and so is solid-liquid separation. Thus, during dewatering, the culture medium is removed from the yeast (for example, by draining the fermentation broth through a filter that retains the yeast), or the yeast is otherwise removed from the culture medium. Common processes for dewatering include centrifugation, filtration, and the use of mechanical pressure. These processes can be used individually or in any combination.

The concentrated yeast produced in accordance with the methods of the invention is itself a finished cosmetic or medical ingredient and may be used in cosmetics or medical compositions without further, or with only minimal, modifications or other composition components. For example, the concentrated yeast can be vacuum-packed or frozen. Alternatively, the yeast may be dried via lyophilization, a “freeze-drying” process, in which the yeast is frozen in a freeze-drying chamber to which a vacuum is applied. The application of a vacuum to the freeze-drying chamber results in sublimation (primary drying) and desorption (secondary drying) of the water from the biomass. However, the present disclosure provides a variety of yeast for finished cosmetic or medical composition wherein the yeast have enhanced properties resulting from processing methods of the invention.

Drying the yeast, either predominantly intact or after homogenizing (lysing and mixing to form a homogenate form), may be a step performed prior to further processing or for use of the yeast in methods and compositions described herein. Drying refers to the removal of free water or surface moisture/water from predominantly intact biomass or the removal of surface water from a slurry of homogenized (e.g., by micronization) biomass. Different textures and dispersion properties can be conferred to cosmetic or medical compositions depending on whether the yeast biomass is dried, and if so, the drying method. Drying the biomass generated from the cultured yeast described herein removes water that may be an undesirable component of finished cosmetic or medical compositions. In some cases, drying the biomass may facilitate a more efficient oil extraction process.

In an aspect, the concentrated yeast is drum dried to a flake form to produce flake. In an aspect, the concentrated yeast is spray or flash dried (i.e., subjected to a pneumatic drying process) to form a powder containing predominantly intact cells to produce powder. In an aspect, oil and/or lipids is extracted from the concentrated yeast to form yeast oil or lipids.

In an aspect, disclosed herein are methods of combining whole yeast organisms and/or a lipid yeast extract, as disclosed herein, with at least one other cosmetic or medical ingredient, as disclosed herein, to form a cosmetic or medical composition. In an aspect, a cosmetic or medical composition formed by the combination of yeast and/or lipid yeast extract comprises at least 1%, at least 5%, at least 10%, at least 25%, or at least 50% w/w yeast or lipid yeast extract, respectively. In an aspect, cosmetic or medical compositions formed as described herein comprise at least 2%, at least 3%, at least 4%, at least 15%, at least 20%, at least 30%, at least 35%, at least 40%, at least 45%, at least 55%, at least 60%, at least 65%, at least 70%, at least 75%, at least 80%, at least 85%, at least 90%, or at least 95% w/w yeast or lipid yeast extract.

In an aspect, a cosmetic or medical composition comprises predominantly intact yeast cells. In an aspect, a cosmetic or medical composition comprises at least 50% intact cells, or at least 60%, at least 70%, or at least 80% intact cells, w/w. In an aspect, a cosmetic or medical composition comprises yeast that has been homogenized to form a whole cell dispersion, but with no extraction of any components of the yeast from the whole cell dispersion.

In an aspect, yeast can be substituted for other components that would otherwise be conventionally included in a cosmetic or medical composition. In an aspect, a cosmetic or medical composition disclosed is free of oil other than oil contributed by the yeast cells and is entrapped therein if the yeast is in an intact cell form.

In an aspect, yeast can be substituted for all or a portion of conventional cosmetic or medical ingredients such as exfoliants, antioxidants, colorants, and the like, to the extent that the components of the yeast replace the corresponding conventional components in like kind, or adequately substitute for the conventional components to impart the desired characteristics to the cosmetic or medical composition.

In an aspect, a lipid yeast extract can be substituted for oils, lipids or fats conventionally used in cosmetic or medical compositions. As described herein, lipids produced by yeast can be tailored by culture conditions or lipid pathway engineering to comprise particular fatty acid components. Thus, lipids generated by yeast disclosed herein can be used to replace conventional cosmetic or medical ingredients such as essential oils, fragrance oils, and the like. In an aspect, a cosmetic or medical composition is free of oil or lipids other than lipids extracted from yeast. As used herein, oil and lipid means the fat compounds of yeast, and may be used interchangeably and are not limited by length of carbon backbone, hydrogenation, number of double bonds in the carbon chains, and understood by those of skill in the art to be characterized as fats, in contrast to compounds such as carbohydrates, proteins or nucleic acids.

Yeast or lipid yeast extract, or a combination of both yeast and lipid yeast extract may be combined with at least one cosmetic or medical ingredient in methods to form cosmetic or medical compositions. Cosmetic or medical ingredients can be selected from conventional cosmetic or medical ingredients suitable for use with the yeast or lipid yeast extract, or both, with regard to the intended use of the composition. Such other cosmetic or medical ingredients include, without limitation, absorbents, abrasives, anticaking agents, antifoaming agents, antibacterial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances and flavor oils, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, soaps, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, vitamin derivatives, and natural extracts.

Essential oils include allspice, amyris, angelica root, anise seed, basil, bay, bergamot, black pepper, cajeput, camphor, cananga, cardamom, carrot seed, cassia, catnip, cedarwood, chamomile, cinnamon bark, cinnamon leaf, citronella java, clary sage, clovebud, coriander, cornmint, cypress, davana, dill seed, elemi, eucalyptus, fennel, fir, frankincense, geranium bourbon, geranium roast, geranium, ginger, grapefruit pink, grapefruit, gurjum balsam, hyssop, juniper berry, lavandin, lavandula, lavender, lemon myrtle, lemon tea tree, lemon, lemongrass, lime, litsea cubeba, mandarin, marjoram, mullein, myrrh, neroli, nerolina, niaouli, nutmeg, orange, palmarosa, patchouli, peppermint, petitgrain, pine needle, ravensara, ravintsara, rosalina, rose, rosemary, rosewood, sage, sandalwood, spearmint, spikenard, star anise, tangerine, tea tree, thyme, tulsi, verbena, vetiver, ylang ylang, and zdravetz, or combinations thereof.

Fragrances and flavor oils include absolute tulip, almond, amaretto, amber, anais, apple, apple cinnamon, apple spice, apricot, apricot creme, arabian musk, asian pear, asian plum blossom, autumn woods, banana, basil, basil nectarine, bay rum, bayberry, bergamot, berries and cream, birthday cake, black cherry, black tea, blackberry tea, blackcurrent, blue nile, blueberry delight, brambleberry preserves, brown sugar, bubble gum, buttercream, butterscotch, calla lily, cantaloupe, caramel apple, carnation, carrot cake, chai tea, chamomile, china musk, china rain, chinese peony, chrysanthemum, cinnamon, coconut, coconut cream, cotton candy, cranberry, cucumber, cucumber melon, daffodil, dandelion, delphinium, dewberry, dulce de leche, earl grey tea, easter cookie, egg nog, egyptian musk, enchanted forest, english lavender, english pear, evergreen, fig, frangipani, frankincense, french vanilla, fresh apple, fresh brewed coffee, fruit punch, gardenia, geranium, ginger lily, gingerbread, grape, grapefruit, green apple, green grass, green tea, guava, guava flower, hawaiian white ginger, heliotrope, hemp, herbaceous, holiday fruitcake, hollyberry, honey ginger, honey, honeysuckle, jasmine, jasmine tea, juniper berries, kiwi, lavender, leather, lemon, lemon parsley, lilac, lime, loganberry, lotus blossom, magnolia, mandarin, mango, mango and kiwi, maple, milk chocolate, mimosa, minty lime, mulberry, myrrh, neroli, oakmoss, oatmeal, ocean rain, orange blossom, orange sherbet, orange vanilla, papaya, passion fruit, patchouli, peach, peaches and cream, pearberry, peppermint, pikaki, pina colada, pineapple, pomegranate, pumpkin pie, raisins and almonds, raspberry, roasted nuts, rosewood, sage, sandalwood, sassafras, sea moss, sesame, siberian pine, snowberry, spanish moss, spice, strawberry, sugar plum, suntan lotion, sweet clove, sweet grass, sweet pea, tangerine, that coconut, timber, tomato leaf, vanilla, watermelon, white chocolate, wild cherry, wisteria, witches brew, and ylang ylang, or combinations thereof.

Exfoliants include particles that can be used to dislodge dead skin cells, dirt, or other materials from the surface of the skin, and include without limitation, fruit seeds and fibers, grain powders, nut and seed meals, and oil or wax beads. Fruit fibers include blueberry, cranberry, grape, kiwi, raspberry, blackberry, strawberry, coffee fruit and the like. Grain powders include oat powder, and almond powder, or the like, milled to varying degrees of coarseness. Polymer beads, such as those made from polyethylene, or the like, can also be used. The removal of dead skin cells and/or the outer most layer of skin can provide an opportunity for bioactive agents, such as carotenoids, which can also be present in the compositions of the invention, to have greater access to deeper layers of the skin.

Cosmetic or medical ingredients may comprise extracts, including herbal extracts derived from conventional extraction procedures, or via the use of liquefied carbon dioxide. Herbs may include, but are not limited to, aloe vera leaf, alfalfa leaf, alkanet root, annatto seed, arrowroot, burdock root, calendula petals, carrot root, chamomile flower, comfrey leaf, cornsilk, dutch blue poppies, fennel seed, ginger root, ginseng, green tea leaf, jasmine flower, juniper berries, lavender buds, lemon peel, lemongrass, marshmallow root, nettles, oat straw, orange peel, paprika, parsley, peppermint leaf, rose buds, rose petals, rosehip, rosemary leaf, shavegrass, spearmint leaf, and St. John's wort, or combinations thereof:

Cosmetic or medical ingredients may comprise colorings, including, but not limited to, glitters, green #5, green #8, orange #4, red #22, red #33, violet #2, blue #1, green #3, red #40, yellow #5, yellow #6, green #6, red #17, iron oxide colorants as well as pearlescent micas and tinting herbs such as henna leaf, sandalwood, turmeric, cranberry, kiwi, raspberry, alkanet, annatto, carrot root, nettles, paprika, and parsley.

Specific examples of other cosmetic or medical ingredients are disclosed herein. Any one or more of these can be optionally combined with yeast or lipid yeast extract or combinations of both, to form a cosmetic or medical composition. The active ingredients disclosed herein are categorized by their cosmetic and/or therapeutic benefit or their postulated mode of action. However, it is to be understood that these ingredients can in some instances provide more than one cosmetic and/or therapeutic benefit or operate via more than one mode of action. Therefore, classifications herein are made for convenience and are not intended to limit the ingredient to that particular application or applications listed.

An anti-inflammatory agent can optionally be added to the compositions of the present invention, preferably from about 0.1% to about 10%, more preferably from about 0.5% to about 5%, of the composition, w/w. An anti-inflammatory agent may enhance the skin appearance, e.g., such agents contribute to a more uniform and acceptable skin tone or color. The exact amount of anti-inflammatory agent to be used in the compositions will depend on the particular anti-inflammatory agent utilized since such agents vary widely in potency, and those of skill in the art can determine such amounts depending on the desired effects of the compositions.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used.

A second class of anti-inflammatory agents which is useful in the compositions includes nonsteroidal anti-inflammatory agents. The variety of compounds encompassed by this group are well-known to those skilled in the art. For detailed disclosure of the chemical structure, synthesis, side effects, etc. of nonsteroidal anti-inflammatory agents, reference may be had to standard texts, including Anti-inflammatory and Anti-Rheumatic Drugs, K. D. Rainsford, Vol. 1-ill, CRC Press, Boca Raton, (1985), and Anti-inflammatory Agents, Chemistry and Pharmacology, 1, R. A. Scherrer, et al., Academic Press, New York (1974), each incorporated herein by reference.

Specific non-steroidal anti-inflammatory agents useful in methods and compositions include, but are not limited to: 1) the oxicams, such as piroxicam, isoxicam, tenoxicam, sudoxicam, and CP-14,304; 2) the salicylates, such as aspirin, disalcid, benorylate, trilisate, safapryn, solprin, diflunisal, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is particularly useful for topical application.

Other anti-inflammatory agents are useful in methods and compositions disclosed herein, Such agents may suitably be obtained as an extract by suitable physical and/or chemical isolation from natural sources (e.g., plants, fungi, or by-products of microorganisms). For example, candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C2-C24 saturated or unsaturated esters of the acids, such as C10-C24, or C16-C24. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

In an aspect, a composition may also optionally comprise a retinoid. Vitamin B3 compounds and retinoids provide benefits in regulating skin condition, especially in therapeutically regulating signs of skin aging, more especially wrinkles, lines, and pores. Without intending to be bound or otherwise limited by theory, it is believed that the vitamin B3 compounds increase the conversion of certain retinoids to trans-retinoic acid, which is believed to be the biologically active form of the retinoid, to provide synergistic regulation of skin condition (namely, increased conversion for retinol, retinol esters, and retinal). In addition, the vitamin B3 compounds unexpectedly mitigate redness, inflammation, dermatitis and the like which may otherwise be associated with topical application of retinoid (often referred to, and hereinafter alternatively referred to as “retinoid dermatitis”). Furthermore, combined vitamin B3 compounds and retinoid(s) tend to increase the amount and activity of thioredoxin, which tends to increase collagen expression levels via the protein AP-1. Compositions disclosed herein may provide reduced active levels, and therefore reduced potential for retinoid dermatitis, while retaining significant positive skin conditioning benefits. In addition, higher levels of retinoid(s) may be used to obtain greater skin conditioning efficacy, without undesirable retinoid dermatitis occurring.

As used herein, “retinoid(s)” includes all natural and/or synthetic analogs of Vitamin A or retinol-like compounds which possess the biological activity of Vitamin A in the skin as well as the geometric isomers and stereoisomers of these compounds. A retinoid may be retinol, retinol esters (e.g., C2-C22 alkyl esters of retinol, including retinyl palmitate, retinyl acetate, retinyl proprionate), retinal, and/or retinoic acid (including all-trans retinoic acid and/or 13-cis-retinoic acid). These compounds are well known in the art and are commercially available from a number of sources, e.g., Sigma Chemical Company (St. Louis, Mo.).

Cosmetic or medical compositions disclosed herein may contain an effective amount of a retinoid, such that the resultant composition is effective for regulating a skin condition, for example, for affecting visible and/or tactile discontinuities in skin, for affecting signs of skin aging, for affecting visible and/or tactile discontinuities in skin texture associated with skin aging. A compositions may comprise from about 0.005% to or about 2%, about 0.01% to about 2%, retinoid, w/w. Retinol may be used in an amount of from about 0.01% to about 0.15% w/w; retinol esters may be used in an amount of from about 0.01% to about 2% w/w (e.g., about 1%); retinoic acids may be used in an amount of from about 0.01% to about 0.25% w/w. The retinoid may be included as the substantially pure material, or as an extract obtained by suitable physical and/or chemical isolation from natural (e.g., plant) sources. The retinoid is preferably substantially pure.

In an aspect, a composition disclosed herein may comprise an antibacterial agent. As used herein, “antibacterial agent” means a compound capable of destroying bacteria cells, preventing the development of bacteria or preventing the pathogenic action of bacteria. Antibacterial agents are useful, for example, in controlling acne. An effective amount of an antibacterial agent can be added to cosmetic or medical compositions of the subject invention, for example, from about 0.001% to about 10%, from about 0.01% to about 5%, from about 0.05% to about 2% or from about 0.05% to about 1% (w/w) of the compositions. Antibacterial agents useful in the cosmetic or medical compositions include, but are not limited to, benzoyl peroxide, erythromycin, tetracycline, clindamycin, azelaic acid, and sulfur resorcinol.

In an aspect, compositions disclosed herein may comprise an anti-androgen compound. As used herein, “anti-androgen” means a compound capable of correcting androgen-related disorders by interfering with the action of androgens at their target organs. A target organ for a disclosed cosmetic or medical compositions can be animal skin, including but not limited to, mammalian skin, hair, nails or other integumentary structures. Exemplary antiandrogens include pregnenalone (and its derivatives), hops extract, oxygenated alkyl substituted bicyclo alkanes (e.g., ethoxyhexyl-bicyclo octanones such as marketed by Chantal Pharmaceutical of Los Angeles, Calif. under the trade names ETHOCYN and CYOCTOL, and 2-(5-ethoxy hept-1-yl)bicylo[3.3.0]octanone), and oleanolic acid. Suitable antiandrogens are disclosed in U.S. Pat. Nos. 4,689,345 and 4,855,322, both issued to Kasha et al. on Aug. 25, 1987 and Aug. 8, 1989, respectively, each incorporated herein by reference. Antiandrogens can optionally be added to cosmetic or medical compositions of the invention.

Exposure to ultraviolet light can result in excessive scaling and texture changes of the stratum corneum. Cosmetic or medical compositions disclosed herein may comprise a sunscreen or sunblock. Suitable sunscreens or sunblocks may be organic or inorganic. A wide variety of conventional sunscreening agents are suitable for use in cosmetic or medical compositions described herein. Sagarin, et al., at Chapter VIII, pages 189 et seq., of Cosmetics Science and Technology (1972), discloses numerous suitable agents, and is incorporated herein by reference. Specific suitable sunscreening agents include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylacetoumbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one) and 4-isopropyl-di-benzoylmethane.

Cosmetic or medical compositions may comprise sunscreens such as those disclosed in U.S. Pat. No. 4,937,370 issued to Sabatelli on Jun. 26, 1990, and U.S. Pat. No. 4,999,186 issued to Sabatelli & Spirnak on Mar. 12, 1991, both of which are incorporated herein by reference, or those sunscreens known to those of skill in the art. The sunscreens disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. Members of this class of sunscreening agents include 4-N,N-(2-ethylhexyl)methyl-aminobenzoic acid ester of 2,4-dihydroxybenzophenone; N,N-di-(2-ethylhexyl)-4-aminobenzoic acid ester with 4-hydroxydibenzoylmethane; 4-N,N-(2-15 ethylhexyl)methyl-aminobenzoic acid ester with 4-hydroxydibenzoylmethane; 4-N,N-(2-ethylhexyl)methyl-aminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N,N-di-(2-ethylhexyl)-4-aminobenzoic acid ester of 4-(2-hydroxyethoxy)dibenzoylmethane and mixtures thereof. Suitable inorganic sunscreens or sunblocks include metal oxides, e.g., zinc oxide and titanium dioxide.

An effective amount of the sunscreen or sunblock is used, typically from about 1% to about 20%, more typically from about 2% to about 10%, w/w. Exact amounts will vary depending upon the sunscreen chosen and the desired Sun Protection Factor (SPF).

Compositions disclosed herein may comprise an agent to improve the skin substantivity of those compositions, particularly to enhance their resistance to being washed off by water, or rubbed off. A substantivity agent which will provide this benefit is a copolymer of ethylene and acrylic acid. Compositions comprising this copolymer are disclosed in U.S. Pat. No. 4,663,157, Brock, issued May 5, 1987, which is incorporated herein by reference.

Cosmetic or medical compositions may comprise an anti-oxidant/radical scavenger as an ingredient. An anti-oxidant/radical scavenger is useful for providing protection against UV radiation which can cause increased scaling or texture changes in the stratum corneum and against other environmental agents which can cause skin damage. An effective amount of an anti-oxidant/radical scavenger may be added to the compositions disclosed herein, for example, from about 0.1% to about 10%, from about 1% to about 5%, (w/w) of the composition.

Anti-oxidants/radical scavengers include, but are not limited to, ascorbic acid (vitamin C) and its salts, ascorbyl esters of fatty acids, ascorbic acid derivatives (e.g., magnesium ascorbyl phosphate), tocopherol (vitamin E), tocopherol sorbate, other esters of tocopherol, butylated hydroxy benzoic acids and their salts, 6-hydroxy-2,5,7,8-tetramethylchroman-2-carboxylic acid (commercially available under the tradename Trolox.sup.R), gallic acid and its alkyl esters, especially propyl gallate, uric acid and its salts and alkyl esters, sorbic acid and its salts, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, lysine, methionine, proline, catalase, superoxide dismutase, lactoferrin, silymarin, tea extracts, grape skin/seed extracts, melanin, and rosemary extracts may be used.

As used herein, “chelating agent” refers to an active agent capable of removing a metal ion from a system by forming a complex so that the metal ion cannot readily participate in or catalyze chemical reactions. The inclusion of a chelating agent may be useful for providing protection against UV radiation which can contribute to excessive scaling or skin texture changes and against other environmental agents which can cause skin damage.

An effective amount of a chelating agent can optionally be added to a cosmetic or medical composition disclosed herein, from about 0.1% to about 10%, from about 1% to about 5%, (w/w) of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995; all incorporated herein by reference. For example, chelators useful in compositions are furildioxime and derivatives thereof.

Compositions of the present invention may comprise an organic hydroxy acid. Suitable hydroxy acids include C1-C18 hydroxy acids, such as C8 or below. The hydroxyl acids can be substituted or unsubstituted, straight chain, branched chain or cyclic (preferably straight chain), and saturated or unsaturated (mono- or poly-unsaturated) (preferably saturated). Non-limiting examples of suitable hydroxy acids include salicylic acid, glycolic acid, lactic acid, 5 octanoyl salicylic acid, hydroxyoctanoic acid, hydroxycaprylic acid, and lanolin fatty acids. Concentrations of the organic hydroxy acid may range from about 0.1% to about 10%, from about 0.2% to about 5%, from about 0.5% to about 2%, w/w. Salicylic acid is an example of an organic hydroxyl acid. For example, organic hydroxy acids tend to improve the texture of the skin. Compositions disclosed herein may comprise a desquamation agent. In an aspect, desquamation agents, which may also be known as exfoliants, can comprise from about 0.1% to about 10%, from about 0.2% to about 5%, or from about 0.5% to about 4% w/w of a cosmetic or medical composition. Desquamation agents tend to improve the texture of the skin (e.g., smoothness). A variety of desquamation agents are known in the art and are suitable for use herein, including but not limited to the organic hydroxy agents described above. For example, enzymatic desquamation agents have been used.

Compositions disclosed herein may comprise an effective amount of a depilation agent. When used, the composition may comprise from about 0.1% to about 10%, from about 0.2% to about 5%, from about 0.5% to about 2% w/w of a depilation agent. A depilation agent may comprise a sulfhydryl compound, e.g., N-acetyl-L-cysteine.

Composition disclosed herein may comprise a skin lightening agent. A compositions may comprise from about 0.1% to about 10%, from about 0.2% to about 5%, from about 0.5% to about 2%, w/w of a skin lightening agent. Suitable skin lightening agents include those known in the art, including kojic acid, arbutin, ascorbic acid and derivatives thereof, e.g., magnesium ascorbyl phosphate.

Compositions disclosed herein may comprise a zinc salt. Zinc salts may be used when the composition contains a sulfhydryl compound, e.g., N-acetyl-L-cysteine. Without intending to be limited or bound by theory, it is believed that the zinc salt acts as a chelating agent capable of complexing with the sulfhydryl compound prior to topical application, stabilizes the sulfhydryl compound and/or controls odor associated with the sulfhydryl compound. Concentrations of the zinc salt can range from about 0.001% to about 10%, from about 0.01% to about 5%, from about 0.1% to about 0.5% by weight of the composition.

Zinc salts include, but are not limited to, zinc acetate, zinc acetate hydrates such as zinc acetate-2-water, zinc aluminum oxide complexes such as gahnite, zinc diamine, zinc antimonide, zinc bromate hydrates such as zinc bromate-6 water, zinc bromide, zinc carbonates such as zincspar and smithsonite, zinc chlorate hydrates such as zinc chlorate-4-water, zinc chloride, zinc diamine dichloride, zinc citrate, zinc chromate, zinc dichromate, zinc diphosphate, zinc hexacyanofluoride ferrate (11), zinc fluoride, zinc fluoride hydrates such as zinc fluoride-4-water, zinc formate, zinc formate hydrates such as zinc formate-2-water, zinc hydroxide, zinc iodate, zinc iodate hydrates such as zinc iodate-2-water, zinc iodide, zinc iron oxide complexes, zinc nitrate hydrates such as zinc nitrate-6-water, zinc nitride, zinc oxalate hydrates such as zinc oxalate-2-water, zinc oxides such as zincite, zinc perchlorate hydrates such as zinc perchlorate-6-water, zinc permanganate hydrates such as zinc permanganate-6-water, zinc peroxide, zinc p-phenolsulfonate hydrates such as zinc p-phenosulfonate-8-water, zinc phosphate, zinc phosphate hydrates such as zinc phosphate-4-water, zinc phosphide, zinc-propionate, zinc selenate hydrates such as zinc selenate-5-water, zinc selenide, zinc silicates such as zinc silicate (2) and zinc silicate (4), zinc silicon oxide water complexes such as hemimorphite, zinc hexafluorosilicate hydrates such as zinc hexafluorosilicate-6-water, zinc stearate, zinc sulfate, zinc sulfate hydrates such as zinc sulfate-7-water, zinc sulfide, zinc sulfite hydrates such as zinc sulfite-2-water, zinc telluride, zinc thiocyanate, zinc (II) salts of N-acetyl L-cysteine, and mixtures thereof.

Compositions disclosed herein may a humectant, moisturizing agent or other skin conditioning agent. A variety of these materials can be employed and each can be present at a level of from or about 0.1% to or about 20%, from or about 1% to or about 10%, or from or about 2% to or about 5%, w/w. These materials include guanidine; glycolic acid and glycolate salts (e.g. ammonium and quaternary alkyl ammonium); lactic acid and lactate salts (e.g. ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol and the like; polyethylene glycols; sugars and starches; sugar and starch derivatives (e.g., alkoxylated glucose); hyaluronic acid; lactamide monoethanolamine; acetamide monoethanolamine; and mixtures thereof. Also useful are the propoxylated glycerols described in U.S. Pat. No. 4,976,953, which is incorporated herein by reference.

Compositions disclosed herein may C1-C30 monoesters and polyesters of sugars and related materials. These esters are derived from a sugar or polyol moiety and one or more carboxylic acid moieties. Depending on the constituent acid and sugar, these esters can be in either liquid or solid form at room temperature. Examples of liquid esters include; glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. A preferred solid material is sucrose polyester in which the degree of esterification is 7-8, and in which the fatty acid moieties are C:18 mono- and/or di-unsaturated and behenic, in a molar ratio of unsaturates:behenic of 1:7 to 3:5. A solid sugar polyester is the octaester of sucrose in which there are about 7 behenic fatty acid moieties and about 1 oleic acid moiety in the molecule. The ester materials are further described in, U.S. Pat. Nos. 2,831,854, 4,005,196, to Jandacek, issued Jan. 25, 1977; U.S. Pat. No. 4,005,195, to Jandacek, issued Jan. 25, 1977, U.S. Pat. No. 5,306,516, to Letton et al., issued Apr. 26, 1994; U.S. Pat. No. 5,306,515, to Letton et al., issued Apr. 26, 1994; U.S. Pat. No. 5,305,514, to Letton et al., issued Apr. 26, 1994; U.S. Pat. No. 4,797,300, to Jandacek et al., issued Jan. 10, 1989; U.S. Pat. No. 3,963,699, to Rizzi et al, issued Jun. 15, 1976; U.S. Pat. No. 4,518,772, to Volpenhein, issued May 21, 1985; and U.S. Pat. No. 4,517,360, to Volpenhein, issued May 21, 1985; all of which are incorporated by reference herein in their entirety.

Compositions disclosed herein may comprise compounds that stimulate the production of collagen. Such compounds include Factor X (kinetin), Factor Z (zeatin), n-methyl taurine, dipalmitoyl hydroxyproline, palmitoyl hydroxyl wheat protein, biopeptide CL (palmitoyl glycyl-histidyl-lysine), ASC III (Amplifier of Synthesis of Collagen III, E. Merck, Germany), beta glucan, and ceramides or the like, for example, ceramide 1-6.

Compositions disclosed herein may an oil absorbent such as are known in the art, e.g. clays (e.g. bentonite) and polymeric absorbents (e.g., Polymeric derivatised starches, (e.g., from National Starch), Derivatised globulin proteins, such as BioPol OE (Arch PC), MICROSPONGES 5647 and POLYTRAP, both commercially available from Advanced Polymer Systems, Inc. of Redwood City, Calif., USA., MICROSPONGES 5647 is a polymer mixture derived from styrene, methyl methacrylate, and hydrogel acrylate/methacrylate.

Compositions disclosed herein may comprise one or more of the following: water-soluble vitamins and derivatives thereof (e.g., vitamin C); polyethyleneglycols and polypropyleneglycols; polymers for aiding the film-forming properties and substantivity of the composition (such as a copolymer of eicosene and vinyl pyrrolidone, an example of which is available from GAF Chemical Corporation as Ganex® V-220). Also useful are crosslinked and noncrosslinked nonionic and cationic polyacrylamides (e.g., Salcare SC92 which has the INCI designation polyquaternium 32 (and) mineral oil, and Salcare SC 95 which has the INCI designation polyquaternium 37 (and) mineral oil (and) PPG-1 trideceth-6, and the nonionic Seppi-Gel polyacrylamides available from Seppic Corp.). Also useful are crosslinked and uncrosslinked carboxylic acid polymers and copolymers such as those containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (examples useful herein include the carbomers, which are homopolymers of acrylic acid crosslinked with allyl ethers of sucrose or pentaerytritol and which are available as the Carbopol® 900 series from B.F. Goodrich, and copolymers of C.sub.10-30 alkyl acrylates with one or more monomers of acrylic acid, methacrylic acid, or one of their short chain (i.e., C.sub.1-4 alcohol) esters, wherein the crosslinking agent is an allyl ether of sucrose or pentaerytritol, these copolymers being known as acrylates/C10-30 alkyl acrylate crosspolymers and are commercially available as Carbopol® 1342, Pemulen TR-1, and Pemulen TR-2, from B.F. Goodrich).

In an aspect, disclosed are cosmetic or medical compositions comprising at least 0.1% w/w yeast or lipid yeast extract, or a combination of both yeast and lipid yeast extract. In an aspect, a cosmetic or medical composition may comprise at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% w/w yeast or lipid yeast extract, or a combination of both yeast and lipid yeast extract. The remainder of a cosmetic or medical composition may comprise water or other conventional cosmetic or medical ingredients, including those identified herein.

Compositions disclosed herein may be in the form of finished cosmetic or medical products for use in skin care, bathing, and/or other applications pertaining to the maintenance or improvement of an individual's appearance or health. In an aspect, compositions disclosed herein are in the form of cosmetic or medical ingredients themselves, for use in combination with other cosmetic or medical ingredients in the production of finished cosmetic or medical products.

In an aspect, compositions disclosed herein may comprise at least 0.1% w/w yeast, or a greater percentage as disclosed herein. The yeast generally comprises at least 0.1% lipid yeast extract by dry weight, and can include greater amounts of lipid yeast extract as well as other constituents as disclosed herein. The yeast useful in the cosmetic or medical compositions of the invention can be derived from one or more species of yeast cultured and/or genetically engineered as described herein.

In an aspect, cosmetic or medical compositions comprising yeast can be formulated as decorative or care cosmetics with one or more other cosmetic or medical ingredients. Exemplary cosmetic or medical compositions include, without limitation, skin-care creams, lotions, powders, perfumes and deodorants, lipsticks, bath oils, bath scrubs and cleansing products, masks, and the like.

In an aspect, cosmetic or medical compositions disclosed herein comprise at least 0.1% w/w lipid yeast extract, or a greater percentage as disclosed herein. The lipid yeast extract is derived from cultures of yeast grown under heterotrophic conditions or those comprising at least 0.1% lipid yeast extract by dry cell weight, as described herein. In an aspect, the yeast can be genetically engineered.

In an aspect, cosmetic or medical compositions comprising lipid yeast extract can be formulated as decorative or care cosmetics with one or more other cosmetic or medical ingredients. Exemplary cosmetic or medical compositions include, without limitation, skin-care creams, lotions, beauty oils, perfumes and deodorants, lipsticks, bath oils, bath scrubs and cleansing products, masks, and the like.

In an aspect, yeast cosmetic or medical compositions in accordance with the present invention can be used in otherwise conventional finished cosmetic or medical products. In these instances, the cosmetic or medical composition comprising yeast or lipid yeast extract, or a combination of both yeast and lipid yeast extract, is combined with one or more other cosmetic or medical ingredients, as described herein, to form a cosmetic or medical composition that may be packaged as a finished cosmetic or medical product. In some cases, yeast cosmetic or medical compositions of the present invention can be packaged as a cosmetic or medical ingredient with optional instructions for combining the yeast composition with conventional cosmetic or medical ingredients to create finished cosmetic or medical products.

In an aspect, the present invention is directed to a method of preparing a finished cosmetic or medical composition, e.g., a skin-care product, comprising (i) culturing a population of yeast under conditions to generate yeast comprising at least 0.10% lipid yeast extract by dry weight, (ii) harvesting the biomass from the yeast culture, (iii) performing one or more optional processing steps, e.g., drying the yeast or extracting lipids from the yeast, (iv) combining the yeast or the lipid yeast extract with at least one other cosmetic or medical ingredient to form a cosmetic or medical composition, and (v) packaging the cosmetic or medical composition with optional instructions for its use as a finished cosmetic or medical product.

The present disclosure comprises methods of using a cosmetic or medical composition comprising yeast or lipid yeast extract, or a combination of both yeast and lipid yeast extract in the manner known for such a cosmetic or medical composition. For example, a cosmetic composition is applied to the outer surface of a subject and allowed to remain on the surface for a determined timeframe. For example, a foundation cream comprising yeast or lipid yeast extract, or a combination of both yeast and lipid yeast extract would be applied to the face of an individual and allowed to remain on the facial skin for as long as the individual desired, such as for 8 hours. A method of the disclosure comprises applying or providing a cosmetic composition comprising a composition comprising yeast or lipid yeast extract, or a combination of both yeast and lipid yeast extract to the skin, such as facial skin, of an individual and maintaining the cosmetic composition on the skin for a predetermined time. A method of the disclosure comprises providing or administering an effective amount of a medical composition comprising yeast or lipid yeast extract, or a combination of both yeast and lipid yeast extract to an individual.

In an aspect, disclosed is a method of using a composition comprising yeast or lipid yeast extract, or a combination of both yeast and lipid yeast extract to soften and impart pliability to skin. In an aspect, the yeast composition comprises predominantly intact yeast cells containing at least 0.1% lipid yeast extract by dry weight. The yeast lipid present in the composition may be encapsulated in cells of the yeast. The yeast composition is applied to human skin and retained in contact with the skin for a period of time sufficient to permit release of a specified percentage of the lipids from the intact yeast cells by enzymatic degradation of the yeast cells. For example, the composition can be retained in contact with the skin for a period of time sufficient to release at least 50% w/w of the lipid yeast extract from the predominantly intact cells. In some cases, this period may be from 1-4 hours.

Without intending to be bound by any particular theory, it is believed that enzymes present on human skin will slowly degrade the intact yeast cells, thereby releasing the intracellular contents, including lipid yeast extract, over a period of time. In an aspect, the yeast composition is retained in contact with the skin for at least 15 minutes, for at least 30 minutes, for at least 45 minutes, for at least 1 hour, for at least 2 hours, for at least 3 hours, or for at least 4 hours or more.

Yeast compositions useful in the method disclosed herein can also comprise cells containing at least 25%, at least 35%, or at least 45% lipids by dry weight. In other cases, the cells may contain other percentages of lipids as described herein. In some cases, mixtures of yeast cells having different lipid profiles can be combined together to form a yeast composition.

In the extraction, both Phosphatidylcholine (PC) and Lysophosphatidylcholine (LPC) were abundant lipids identified. PC can be used in personal care as an emulsifier, as an epidermal barrier constituent, and essential to the creation of delivery vehicles (Liposomes), the identification of LPC leads to many other applications. For example, in topical products LPC could have application in skin cancer.

Furthermore, pharmaceutical compositions of LPC can be used in antitumor treatments. LPC selectively targets plasma membrane of tumor cells to signal apoptosis. These yeast cells have defense mechanisms that can be utilized for many applications and extracting the inherent antibiotics the cells produce can lead to many applications. A compound that could be cephalosporin was identified. Certain phospholipids may have anti-viral activity or be made into anti-viral analogs.

Since yeast cell extract also comprises amino acids or polypeptides, there may be peptides and enzymes involved in signaling.

Methods may comprise anti-tumor and anti-proliferative phospholipids. There may be references that further show the value of producing a comprehensive mixture of phospholipids for select optimization and utilization pf phospholipids for this application. It was demonstrated that extracts of the yeast are capable of forming vesicles when using sonification.

Methods for immunomodulation may comprise phospholipids disclosed herein.

Phospholipid amino acid complexes may be used in nutritional foods and beverages. Riboflavin and Pyruvates are involved in the production of ATP (Kreb's cycle). Delivery of these with phospholipids comprise performance enhancer products for nutritional supplements and functional beverages.

All references cited herein, including patents, patent applications, and publications, are hereby incorporated by reference in their entireties, whether previously specifically incorporated or not. The publications mentioned herein are cited for the purpose of describing and disclosing reagents, methodologies and concepts that may be used in connection with the present invention. Nothing herein is to be construed as an admission that these references are prior art in relation to the inventions described herein.

Although this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used with reference to a nucleic acid, “active in yeast” refers to a nucleic acid that is functional in yeast. For example, a promoter that has been used to drive an antibiotic resistance gene to impart antibiotic resistance to a transgenic yeast is active in yeast. Examples of promoters active in yeast are promoters endogenous to certain algae species and promoters found in plant viruses.

As used in the specification and the appended claims, the singular forms “a,” “an” and “the” include plural referents unless the context clearly dictates otherwise. Thus, for example, reference to “a methylation site,” “an array,” or “the patient” includes mixtures of two or more such methylation sites, arrays, or patients, and the like.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

As used herein, the terms “optional” or “optionally” mean that the subsequently described event or circumstance may or may not occur and that the description includes instances where said event or circumstance occurs and instances where it does not.

“Axenic” means a culture of an organism that is free from contamination by other living organisms.

“Bioreactor” means an enclosure or partial enclosure in which cells are cultured, optionally in suspension.

The term “co-culture”, and variants thereof such as “co-cultivate”, refer to the presence of two or more types of cells in the same bioreactor. The two or more types of cells may both be microorganisms, such as yeast, or may be a yeast cell cultured with a different cell type. The culture conditions may be those that foster growth and/or propagation of the two or more cell types or those that facilitate growth and/or proliferation of one, or a subset, of the two or more cells while maintaining cellular growth for the remainder.

As used herein, “cosmetic or medical ingredient” means an ingredient conventionally used in cosmetic or medical products that is not physically or chemically incompatible with the yeast components described herein. “Cosmetic or medical ingredients” include, without limitation, absorbents, abrasives, anticaking agents, antifoaming agents, antimicrobial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins and derivatives thereof, and natural extracts. Such “cosmetic or medical ingredients” are known in the art. Nonexclusive examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993).

The term “cultivated”, and variants thereof, refer to the intentional fostering of growth (increases in cell size, cellular contents, and/or cellular activity) and/or propagation (increases in cell numbers via mitosis) of one or more cells by use of intended culture conditions. The combination of both growth and propagation may be termed proliferation. The one or more cells may be those of a microorganism, such as yeast. Examples of intended conditions include the use of a defined medium (with known characteristics such as pH, ionic strength, and carbon source), specified temperature, oxygen tension, carbon dioxide levels, and growth in a bioreactor.

As used herein, the term “cytolysis” refers to the lysis of cells in a hypotonic environment. Cytolysis is caused by excessive osmosis, or movement of water, towards the inside of a cell (hyperhydration). The cell cannot withstand the osmotic pressure of the water inside, and so it explodes.

“Dispersion” refers to a distribution of particles more or less evenly throughout a medium, including a liquid or gas. One common form of dispersion is an emulsion made up of a mixture of two or more immiscible liquids such as oil and water.

As used herein, the terms “dry weight” or “dry cell weight” refer to weight as determined in the relative absence of water. For example, reference to a component of yeast as comprising a specified percentage by dry weight means that the percentage is calculated based on the weight of the biomass after all or substantially all water has been removed.

“Exogenously provided” describes a molecule provided to the culture media of a cell culture.

“Lipid profile” refers to the distribution of different carbon chain lengths and saturation levels of glycerolipids in a particular sample of biomass or lipids. For example, a sample could contain glycerolipids in which approximately 60% w/w of the glycerolipid is C18:1, 20% is C18:0, 15% is C16:0, and 5% is C14:0. In cases in which a carbon length is referenced generically, such as “C:18”, such reference can include any amount of saturation; for example, yeast that contains 20% w/w lipid as C:18 can include C18:0, C18:1, C18:2, and the like, in equal or varying amounts, the sum of which constitute 20% w/w of the biomass.

“Homogenate” means biomass that has been physically disrupted.

“Homogenize” means to blend a substance, for example, yeast cells into a homogenous or uniform mixture. In an aspect, a homogenate is created from lysed yeast cells or the lipid yeast extract. In an aspect, the biomass of yeast cells is predominantly intact, but homogeneously distributed throughout the mixture.

As used herein, the phrase “increase lipid yield” refers to an increase in the productivity of a yeast culture by, for example, increasing dry weight of cells per liter of culture, increasing the percentage of cells that constitute lipid, or increasing the overall amount of lipid per liter of culture volume per unit time.

The term “in situ” means “in place” or “in its original position”. For example, a culture may contain a first yeast secreting a catalyst and a second microorganism secreting a substrate, wherein the first and second cell types produce the components necessary for a particular chemical reaction to occur in situ in the co-culture without requiring further separation or processing of the materials.

“Lipids” are a class of molecules that are soluble in nonpolar solvents (such as ether and hexane) and are relatively or completely insoluble in water. Lipid molecules have these properties because they consist largely of long hydrocarbon tails which are hydrophobic in nature. Examples of lipids include fatty acids (saturated and unsaturated); glycerides or glycerolipids (such as monoglycerides, diglycerides, triglycerides or neutral fats, and phosphoglycerides or glycerophospholipids); nonglycerides (sphingolipids, tocopherols, tocotrienols, sterol lipids including cholesterol and steroid hormones, prenol lipids including terpenoids, fatty alcohols, waxes, and polyketides); and complex lipid derivatives (sugar-linked lipids, or glycolipids, and protein-linked lipids). Lipid and oil may be used interchangeably herein and are generally referring to those compounds characterized as fats.

As used herein, the term “lysate” refers to a solution containing the contents of lysed cells.

As used herein, the term “lysis” refers to the breakage of the plasma membrane and optionally the cell wall of a biological organism sufficient to release at least some intracellular content, often by mechanical, viral or osmotic mechanisms that compromise its integrity.

As used herein, the term “lysing” refers to disrupting the cellular membrane and optionally the cell wall of a biological organism or cell sufficient to release at least some intracellular content.

As used herein, the term “osmotic shock” refers to the rupture of cells in a solution following a sudden reduction in osmotic pressure. Osmotic shock is sometimes induced to release cellular components of such cells into a solution.

As used herein, a “polysaccharide-degrading enzyme” refers to any enzyme capable of catalyzing the hydrolysis, or depolymerization, of any polysaccharide. For example, cellulases catalyze the hydrolysis of cellulose.

“Polysaccharides” (also called “glycans”) are carbohydrates made up of monosaccharides joined together by glycosidic linkages. Cellulose is an example of a polysaccharide that makes up certain plant cell walls. Cellulose can be depolymerized by enzymes to yield monosaccharides such as xylose and glucose, as well as larger disaccharides and oligosaccharides.

As used herein, “predominantly intact cells” refers to a population of cells which comprise more than 50%, 75%, or 90% w/w intact cells. “Intact” refers to the physical continuity of the cellular membrane enclosing the intracellular components of the cell and means that the cellular membrane has not been disrupted in any manner that would release the intracellular components of the cell to an extent that exceeds the permeability of the cellular membrane under conventional culture conditions or those culture conditions described herein.

As used herein, the term “sonication” refers to a process of disrupting biological materials, such as a cell, by use of sound wave energy.

Reference to proportions by volume, i.e., “v/v,” means the ratio of the volume of one substance or composition to the volume of a second substance or composition. For example, reference to a composition that comprises 5% v/v lipid yeast extract and at least one other cosmetic or medical ingredient means that 5% of the composition's volume is composed of lipid yeast extract; e.g., a composition having a volume of 100 mm3 would contain 5 mm3 of lipid yeast extract and 95 mm3 of other constituents.

Reference to proportions by weight, i.e., “w/w.” means the ratio of the weight of one substance or composition to the weight of a second substance or composition. For example, reference to a cosmetic or medical composition that comprises 5% w/w yeast and at least one other cosmetic or medical ingredient means that 5% of the cosmetic or medical composition is composed of yeast; e.g., a 100 mg cosmetic or medical composition would contain 5 mg of yeast and 95 mg of other constituents. One of skill in the art can determine whether percentages of components of compositions are w/w or v/v.

### EXAMPLES

### Example 1

Introduction

Debaryomyces (Torulaspora) hansenii is a type of yeast that can tolerate and survive changes in sugar, salt and dryness. It is non-pathogenic and found in water with salt concentration of up to 24% w/w (Breuer and Harms, 2006). It is also found in the cheese and sausages industries (Fleet, 1990; Dalton et al., 1984). D. hansenii is able to eliminate competition by other yeasts due to its ability to tolerate salt and reproduce at low temperatures.

Molecular genetic studies for D. hansenii are still in their infancy. There were 46 gene entries corresponding to 28 different proteins in Genbank before release of the whole genome data. The whole genome is available at the ncbi/nlm.nih website. D. hansenii defines now one of the four clades which constitute this genus. The species contains two varieties, var. hansenii and var. fabryi, the second of them is not very often found and is poorly characterized (Kurtzman and Robnett, 1998).

The yeast D. hansenii uses glucose as a substrate at a very slow rate with typical times for culture reported as 21-28 days. The most common lipids produced are triglycerides, free fatty acids, phosphatidylserine and phosphatidylethanolamine (Merdinger and Frye, 1966).

Commercial Applications

D. hansenii osmotolerance is highly advantageous for some biotechnological applications because it allows quasi-non-sterile production and high product/educt concentrations, conditions which can reduce production costs dramatically. The extreme capacity of D. hansenii to synthesize, accumulate and store lipids is advantageous for the biotechnological production of natural and artificial products. The ability to produce phospholipids can be modified by changing the amount of salt in the culture media allowing for more selective production of products, liposomes of small size can be produced by sonication of lipid fractions.

Odorless culture and production. Members of the genus Debaryomyces are characterized physiologically by their weak or nonexistent fermentation capacities. D. hansenii is able to use alkanes as a food source. This application is useful as a lot of by-products from mining and cracking are alkanes. This particular strain is able to assimilate a large number of sugar substrates such as sucrose, galactose, lactose, mannose, maltose and treehalose among others D. hansenii appears to have a very high coding capacity reflected in 79.2% of its genome, with 6,906 detected coding sequences or (CDs). This characteristic allows this yeast to be used in biotechnological applications. The most abundant solute produced by the yeast is glycerol and it has the capacity to regulate its glycerol metabolism under hyperosmolaric conditions. It can also produce xylitol.

Expressing the genes conferring salt resistance in D. hansenii in plants is an effective strategy to grow crops in arid regions and can make a substantial contribution to reducing hunger in the world. The yeast can also produce D-Arabinitol after the growth phase in batch culture, simultaneously with the excretion of riboflavin. Pyruvic acid can be widely used in the chemical, pharmaceutical and agrochemical industries and the biotechnological production of this acid is a viable alternative to the current chemical method, because it is a relatively cheap, one-step procedure.

D. hansenii also produces important enzymes with commercial applications such as β-glucosidases and superoxide dismutase. This yeast can also produce alkali-soluble glucans that can be used as thickening agents, fat substitutes or sources of dietary fiber. Furthermore, they have antitumor activity, stimulate the immune system and can lower the serum cholesterol levels.

Materials and Methods

Lipid Extractions

D. hansenii (NRRL-Y-1448) (ATCC 10619) was reconstituted by breaking the outer glass vial and carefully removing the cotton plug of the inner vial. The yeast was rehydrated with 400 μl of sterile water and transferred to a sterile 15 ml conical tube (Corning) where it was left overnight at room temperature.

Aliquots of yeast were prepared for inoculation of media.

100 μl aliquots of rehydrated yeast were grown in 9 ml tubes containing Sabourad media (VWR) and at different temperatures (37° C., 24° C. and 28° C.) in a rotary shaker. Volume was added with a sterile pipette and an automated pipettor. Stocks of yeast were grown in 10 cm diameter Sabaourad agar plates (VWR) at room temperature. Optimum density was observed after 20 days. Yeast began lipid production after 24 hours as evidenced by a ring of waxy/fatty material around the top of the culture. The cultures were combined and centrifuged to isolate the yeast and the yield of the culture was calculated. 3.66 g of D. hansenii were harvested and 1.98 g were originally inoculated. % yield in culture: 3.56/1.98*100=179.80% w/w.

100 ml supernatant were collected from the lysed, sonicated cells and 50 ml HPLC grade methanol were added to the supernatant and stirred overnight to dissolve the phospholipids. 100 ml chloroform were added to this supernatant and stirred for 1 hour. The phases were allowed to separate overnight in a 250 ml graduated cylinder. The precipitated cells were saved at 4° C. overnight according to Turk, 2004. The fatty material was separated from the clear liquid and the supernatant. Out of 100 ml of supernatant, 10 ml of fatty material were recovered. Out of 10 ml of dissolved sonicated cells, about 5 ml of fatty material were recovered with about 2.5 ml of white fat and cell debris (top layer) and about 5.0 ml of fatty material and the remaining solvent. The 10 mL of fatty material is a lipid yeast extract of the present disclosure.

Thin Layer Chromatography (TLC)

TLC plates (silica, Whatman LK5 equivalent with glass backing) were pre-washed to remove any UV fluorescent material by migration up to 1 cm from the top in a clean tank containing chloroform/methanol (1/1, v/v). The pre-wash step lasted 1.5 hours. The solvent level was marked on the plate with a pencil. Plates were air-dried in a fume hood for five minutes and placed in a drying rack until used. Immediately before use, plates were completely wetted using a plastic bottle to spray (VWR) with boric acid solution prepared by dissolving 2.3 g of boric acid in 100 ml ethanol. The plates were drained for 5 minutes in a fume hood and dried in a model 10 oven for 15 min at 100 C.

Lipid samples 100 μl chloroform/methanol solution (2:1 v/v) containing 20-200 μg phospholipids were rapidly deposited on plates at 1 cm parallel in the concentration zone. The solvent was allowed to dry (a precaution used to avoid distorted spots) and the plates were rapidly placed in the chromatography tank containing chloroform/ethanol/water/triethylamine (30/35/7/35, v/v). The migration time was 2 hours. The solvent was allowed to reach the 1 cm mark at the top.

Plates were dried in a fume hood (2-5 min max) and sprayed with primuline solution (yellow) made by dissolving 5 mg of primuline in 80/20 acetone/water. After viewing under UV light, photographs were taken and the contour of each spot was outlined. The fluorescent spots, indicating lipids, were scraped from the silica into glass tubes for further analysis by GC-MS.

As described by Vaden et al. in 2005, the neutral lipids migrated with the solvent (seen as a bright line on the top of FIG. 4). Polar lipids (phospholipids) remained at the bottom of the TLC plate. Triacylglycerols (nonpolar) migrated faster than phospholipids. Actual photographs (under UV light) of the plate, revealed primuline staining (fluorescent). Although the spots are difficult to visualize due to the background fluorescence of the TLC plates, the contour of each phospholipid was outlined.

Rf values were calculated and shown in Table 1. The Rf values were in good agreement with the values reported by Leray and Pelletier, 1987.

| | | Distance | Calcu- | |
|---|---|---|---|---|
| | | migrated | lated | Reported |
| | Distance | by | Rf | Rf |
| Spot | migrated | solvent | value | value¹ |
| PE | 3.5 | 7.6 | 0.46 | 0.51 |
| Phosphytidylethanolamine | | | | |
| PS | 3 | 7.6 | 0.39 | 0.3 |
| Phosphytidylserine | | | | |
| PI | 2.3 | 7.6 | 0.30 | 0.26 |
| Phosphytidylinositol | | | | |
| PC | 1.5 | 7.6 | 0.20 | 0.21 |
| Phosphytidylcholine | | | | |

¹Leray and Pelletier, 1987

Results

The inoculated yeast doubled its mass in 20 days at 28° C. and a pH of around 5.6. The yeast can also be grown at room temperature but growth was slower. Attempts to grow yeast at temperatures higher than 28° C. failed even though it has been reported in the literature that the yeast can be grown at 30 and 31° C. (Merdinger and Frye, 1966). Yield of fatty material was high compared to the actual volume of the initial cultures. Increasing the pH has been reported as useful in doubling time for these cells. The doubling time of the cells was 9.2 h at pH 4.0, 2 h at pH 6.0 and 6 h at pH 8.0 (Turk et al., 2007).

Yield of fatty material from supernatant=(10 ml/100 ml)*100=10% w/w. Yield of fatty material from the sonicated cells=(5 ml/10 ml)*100=50% w/w.

TLC analysis for extracellular lipids of the cell-free supernatant was negative, consistent with the results obtained in 1966 by Merdinger and Frye. TLC analysis of the bottom layer of the sonicated cell extract was negative. TLC analysis of the top layer of the sonicated cell extract fatty material was positive. Only four distinct spots were able to be identified: The largest spot (and therefore the largest amount) corresponds to PC and LPC followed by PS and PE in a lower proportion. There was also a small spot at the base of the PS spot, which according to Vaden et al., 2005, corresponds to PI.

Sonication has been reported to induce the formation of small liposomes (Szoka F and Papahadjopoulos, 1980). In breaking the extremophile yeast cell by sonication, liposomes are formed. These liposome vesicles were used in compositions and methods disclosed herein.

As this protocol was adapted to separate the phospholipids of interest, neutral lipids and triacylglycerols were observed to migrate as expected with the solvent as a bright line at the top of the plate. As described by Leray and Pelletier in 1987, poor separation of PS, PE, PI and PC was observed using the normal TLC protocol. The use of boric acid improved the resolution of the spots but it can be necessary to run a two dimensional TLC in order to get better separation of the spots.

Other fluorescent compounds can interfere with the quality of images obtained under UV. Proteins and peptides, with aromatic amino acids are intrinsically fluorescent when excited with UV light. Many enzymatic cofactors, such as FMN, FAD, NAD and porphyrins, are also intrinsically fluorescent under UV light. In order to obtain better graphics, sulfuric acid or iodine can be used to visualize the spots as these methods do not require UV light to reveal spots.

The results obtained demonstrated that it was possible to culture D. hansenii obtained commercially and scale up its production in Sabaourad media with minimum requirements for the culture. For the variety used, the optimum conditions appeared to be 28° C., salt concentration of 2% w/w and pH 5.6 to 6.2. Salt concentration can be varied to increase the production of the phospholipids of interest.

The cultures did not present any fermentation or sulfur odor. However, a mild odor was detected after 3 months.

As D. hansenii is a halophile that grows at 2% w/w salt concentration, there was no contamination in the cultures. In addition, D. hansenii produces toxins that out-compete other yeasts. The use of D. hansenii in the cheese and meat industries indicates that it is safe to use in commercial applications.

The cultures were a milky tan color. There was no need to remove pigments. There was no gas detected as being produced. Moreover, there was no foam produced in the cultures.

Lipids were extracted using an aqueous/organic extraction procedure. It was possible to separate lipids using one dimensional TLC. However, 2D-TLC is recommended to obtain more accurate results and for quantitation. These results indicated that 50% w/w of the pelleted (wet) cells have the potential to yield phospholipids. Merdinger and Devine (1965) reported that neutral lipids comprised 67%, and phospholipids comprised 33%, w/w, of the total lipids isolated from D. hansenii. Lipids extracted from D. hansenii are detailed in PCT/US2014/062464, which is herein incorporated in its entirety.

Though sonication is disclosed herein in Example 1, other methods for disrupting yeast may be used. Autolysis. A yeast suspension is mixed with toluene (or compounds such as ammonium hydroxide) and incubated at room temperature for 24-48 h. The toluene prevents bacterial growth and permeabilizes the yeast membrane. The latter releases a wide variety of hydrolases that attack the cell wall. Homogenization. The homogenizers can be used to disrupt yeast cells. The presses lyse cells by pressurizing the cell suspension and suddenly releasing the pressure. This creates a liquid shear capable of lysing cells. Typical operating pressures for the older type of homogenizers, the French press and Manton-Gaulin homogenizer, are 6000-10,000 psi. Multiple (at least 3) passes are required to achieve a reasonable degree of lysis. The high operating pressures, however, result in a rise in operating temperatures. Therefore, pressure cells are cooled (4° C.) prior to use. In addition to temperature control, care should be taken to avoid inactivating proteins by foaming. Modern homogenizers are more suited to lyze yeast cells since they can be operated at much higher pressures. Glass bead vortexing. Probably the most widely used method is the disruption of yeast cells by agitation with glass beads (0.4-0.5 mm). The simplest method for agitating the glass beads is with the use of a vortex mixer. Several cycles of agitation (30-60 sec) must be interspersed with cycles of cooling on ice to avoid overheating of the cell suspension. Breakage is variable, but can be well over 50% (up to 95%). Above the method is described for small volumes (up to 15 ml) but it can be scaled up to many liters using specialized apparatus. Enzymatic lysis. The enzymatic lysis of yeast cells is based on the digestion of the cell wall by a number of enzymes, of which zymolase and lyticase are the most widely used. The procedure yields spheroplasts that can be prepared and purified as an intermediary step or lysis can be carried out directly. The procedure can be used on any scale but for large scale preparations the enzymes may be expensive. Freezing and grinding. An alternative lysis method is to freeze the cells directly in liquid nitrogen and ground the frozen cells to a powder using a mortar and pestle that are chilled with liquid nitrogen. The powder can be stored indefinitely at −80° C. and the cell lysate can be prepared by adding the powder to 5 volumes of buffer.

The yeast cells are grown in culture, removed from the media and pelleted by centrifugation. After resuspension in saline, the cells are lysed by sonication so that liposomes are formed by the lipids. The lipid components are separated by chloroform/methanol extraction as is known by those of skill in the art. The extracted lipid components are primarily in the form of liposomes. To remove any remaining chloroform/methanol, the extracted lipid components are placed in a rotary evaporator at 30-40° C., for about 8-10 hours per liter of extracted lipid components. This results in a lipid paste. This lipid paste is the yeast lipid extract disclosed herein.

### Example 2

Production of Liposomes from Yeast Lipid Extract

Sample Preparation for Selection of Sonication Candidate:

Initial yeast lipid extract produced had a viscosity range of 5,000-50,000 cps, at times a paste. Initial yeast lipid extract samples were prepare by evaluating 3 different aqueous dilutions

Sample #

1—Yeast Lipid Extract 50%+Distilled water 50%

2—Yeast Lipid Extract 30.0%+Distilled Water 70%

3—Yeast Lipid Extract 20.0%+Distilled Water 80%

Each was placed in a volumetric flask and attached to a wrist action shaker (Burrel Model 75). The shaker lever arm was set to 10 for a period of time of 1 hour at RT 25° C. Upon completion all three samples were observed to display a milky colloidal dispersion and emulsification—with viscosity under 1000 cps and characteristic of milk. The three samples were transferred to sterile 20 ml scintillation vials and placed 10° C. for a period of time of 24 h, and thereafter retrieved and allowed to reach RT 25° C. for visual and microscopic evaluation.

After naked eye visual and microscopic evaluation and although none of the samples experience gross separation, sample #1 was determined to possess the best Tyndall effect as a light scattering colloidal parameter suggesting excellent micelle formation after shaker procedure. This was microscopically confirmed. Hence, the milky luminescent appearance of sample #1 confirmed emulsification properties of yeast lipid extract with 50% distilled water.

Thereafter sample #1 was selected to undergo sonication procedure to assess production of liposomes.

Sonication of Sample #1

Equipment

Q Sonicator Model Q 700

Microtip Probe (⅛″ #4422, Cole Parmer)

Sample #1 was placed into an sterile container and placed into a cold water bath (5° C.) to avoid overheating and maintain condition conducive for cavitation). Sonication conditions were established at 10 minute run time using amplitude of 2 with power range of 12-17, consuming energy ranging from 1,300 to 9.400 Joules. After sonication sample #1 was maintained at 5° C. until image analysis evaluation.

### Example 3

Image Validation of Liposomes Produced by Yeast Lipid Extract Using Transmission Electron Cryo-Microscopy (Cryo-TEM) Imaging

Sample Preparation

The sample was preserved at full concentration in vitrified ice supported by holey carbon films on 400-mesh copper grids. The sample was prepared by applying a 3 μl drop of sample suspension to a cleaned grid, blotting away with filter paper, and immediately proceeding with vitrification in liquid ethane. Grids were stored under liquid nitrogen until transferred to the electron microscope for imaging. Electron microscopy was performed using an FEI Tecnai T12 electron microscope, operating at 120 keV equipped with an FEI Eagle 4k×4k CCD camera. Vitreous ice grids were transferred into the electron microscope using a cryostage that maintains the grids at a temperature below −170° C.

Images of each grid were acquired at multiple scales to assess the overall distribution of the specimen. After identifying potentially suitable target areas for imaging at lower magnifications, high magnification images were acquired at nominal magnifications of 52,000× (0.21 nm/pixel) and 21,000× (0.50 nm/pixel). The images were acquired at a nominal underfocus of −5 μm to −3 μm and electron doses of ˜10-25 e⁻/Å².

Observation

Liposome vesicles with a unilamellar and multi-lamellar lipid bilayer were present in the sample. These ranged in size from 60-350 nm in diameter with bilayer widths from 7-9 nm.

### Example 4

As referred to herein in this and the following Examples, “yeast cell” is intended to mean cells of D. hansenii, and “yeast lipid extract” is the lipid extract of D. hansenii yeast cells using the process of Example 1.

Yeast Lipid Extract Protection Against Skin Cell (Fibroblast) Dehydration

Effect of 3 Test Materials on Dehydration in Human Dermal Fibroblast Cultures, Compared to Petroleum Jelly.

Objective—Skin aging is associated with dehydration due to progressive loss of the barrier function. Among protective ointments developed to counteract this process petroleum jelly is an FDA-recognized benchmark. This project aimed at assessing the protective effect of the test materials listed in Table I in the human dermal fibroblast culture dehydration model, in vitro, as compared to petroleum jelly (Vaseline).

Test Material

3A—Yeast Lipid Extract

3B—Yeast Lipid Extract (50/50 Aqueous solution)

3C—Soybean Lipid Extract (Lysofix—from Kemin)

Materials & Methods

Neonatal human dermal fibroblasts were grown in DMEM/5% FBS w/o Phenol Red to sub-confluence, afterward the cell growth medium was replaced with different experimental conditions (diluted in DMSO; see Table II). After a 15-minute incubation, all fluids were removed (except the non-dehydrated controls) and all cells were exposed to air for 15 min. At the end of the air exposure, cell growth medium was added back and cells were returned to the incubator. After 2 hours of recovery, cell viability was determined by counting cell numbers using sulforhodamine B method (Voigt, 2005). The ensuing colorimetric signal was quantified with Molecular Devices microplate spectrophotometer MAX 190 at 560 nm.

Two experiments were performed, each in 3 or more identical replicates. Statistical significance was calculated with the double-tailed t-test and the p value threshold was set at 0.01.

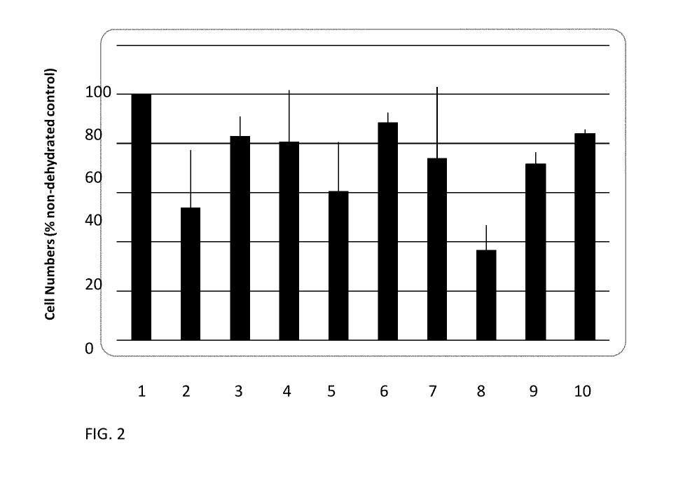

Table 2 shows the effect of pre-treatment of human dermal fibroblasts under differing experimental conditions (before dehydration) on cell viability, and its translation into hydration protection (as standardized to the non-dehydrated control). FIG. 2 is a graphic representation of each test, as numbered and described in Table 2.

TABLE 2

| | | | % Hydration | |
|---|---|---|---|---|
| Pre-treatment | % Cell viability | p value (vs. | protection vs. | |
| condition | vs. non- | non- | non- | No. of |
| (before | dehydrated | dehydrated | dehydrated | bar in |
| dehydration) | control | control) | control | FIG. 2 |
| Air | 54 | 0.001 | 0 | 2 |
| Vaseline 4% | 72 | 0.014 | 38 | 9 |
| 03A 4% | 83 | 0.228 | 63 | 2 |
| 03B 4% | 61 | 0.048 | 14 | 5 |
| 03C 4% | 74 | 0.389 | 43 | 7 |
| Vaseline (10%) | 84 | 0.106 | 65 | 10 |
| 03A 10% | 81 | 0.431 | 58 | 4 |
| 03B 10% | 89 | 0.247 | 75 | 6 |
| 03C 10% | 37 | 0.000 | 0 | 8 |
| no | 100 | 0.000 | 100 | 1 |
| dehydratation | (CONTROL) | | (CONTROL) | |

Results

As illustrated on Table 2 and FIG. 2, the results are as follows:

1. Exposure to air resulted in the average loss of nearly half of the fibroblast population. This decrease of skin cell viability was statistically significant (p<0.01).

2. The pre-treatment of fibroblasts with 4% and 10% of 3A provided a statistically significant protection from dehydration. At 4%, this protection was better than the equivalent dose of the positive control (Vaseline, Personal Care Products, LLC).

3. The pre-treatment of fibroblasts with 4% of 3B provided a mild, non-statistically significant protection from dehydration. At 10%, this protection was significantly better and was better than the equivalent dose of the petroleum jelly.

4. The pre-treatment of fibroblasts with 4% of 3C provided a statistically significant protection from dehydration comparable with the positive control at equivalent concentration. At 10%, this protection was lost due to cytotoxicity.

5. The positive control (petroleum jelly) provided a dose-dependent, statistically-significant protection from dehydration-caused loss of cell viability, technically validating the experiment.

Conclusion

Among the tested experimental materials, 3A seemed to be the best-performing product. See Voigt W. Sulforhodamine B assay and chemosensitivity. Methods Mol Med. 2005; 110:39-48.

### Example 5

Preparation of Yeast Lipid Extract Antioxidant Base
Ingredients in the composition

| | |
|---|---|
| Polysorbate 20 | 0.50% |
| Oxynex K | 0.50% |
| BHA | 0.50% |
| Vitamin E TPGS | 0.70% |
| Tocopherol (Vit E) | 2.70% |
| Tetra Hexadecyl Ascobate | 0.200% |
| Glyceryl Cocoate | 22.00% |
| Caprilic Capric Glycerides | q.s. to 100% |

Procedure: Add all with moderate lightning mixing speed. Begin to heat to 65° C. with continued mixing for 10 minutes and cool to 25° C. and stop. Addition of individual or combination of Yeast Whole Cell or Yeast Lipid Extract A1-A4 is made at room temperature with homogenous mixing in ratio maximum of 50/50 Yeast Whole Cell or Yeast Lipid Extract to the Antioxidant base to improve stability of Yeast Whole Cell or Yeast Lipid Extract. Evaluations using accelerated protocol of 3 months at 5, 25, and 40° C. versus controls using water and mineral oil show color, odor and appearance is maintained acceptable using Antioxidant Base.

### Example 6

Anti-Aging Night Cream with Yeast Lipid Extract
Ingredients in the composition

| Phase A | |
|---|---|
| Distilled Water | q.s to 100% |
| Sodium Metabisulfite | 0.04% |
| Glycerin | 3.00% |
| Modified Potato Starch | 2.50% |
| Phase B | |
| Linoleic Acid | 1.20% |
| Caprilic Capric Triglycerides | 4.20% |
| Glyceryl Stearate Citrate | 2.70% |
| Rosehip Seed Oil | 3.00% |
| Pentaerythrityl Tetraisostearate | 1.70% |
| Polysorbate 80 | 0.60% |
| Cetearyl Alcohol | 1.20% |
| Beeswax | 1.00% |
| Glyceryl Behenate | 0.75% |
| Phase C | |
| Resveratrol | 0.55% |
| Phenoxyethanol | 0.20% |
| Hyaluronic Acid | 2.50% |
| Dipalmitoyl Hydroxyproline | 0.95% |
| Yeast Lipid Extract | 2.75% |
| Retinol 10% in Caprilic Triglycerides | 1.50% |
| Fragrance | 0.20% |

Processing: Add Phase A one by one with moderate lightning mixing until homogenous solution is attained. Then heat to 80° C. with continued mixing. Add all Phase B ingredients, heat to 80° C. with lightening mixing at slow speed. Emulsify by adding Phase B to Phase A and continue lightening mixing at moderate speed. Begin to cool to 35° C. and add Phase C one by one to main batch. Mix for 15 minutes, cool to 25° C. and stop.

### Example 7

Yeast Lipid Extract Reduction of Retinol Induced Irritation

10 woman ages 30-50 with Type I, II skin, self-classified as having sensitive skin and known to be Retinol irritation prone are evaluated after 7 days of product usage for signs of irritation. They are given random blinded samples of Anti-Aging Night Cream with Yeast Lipid Extract versus control devoid of Yeast Lipid Extract. A total of 5 women using Anti-Aging Night Cream with no Yeast Lipid Extract experience moderate irritation inherent to Retinol formulations. A total of 5 women using Anti-Aging Night Cream with Yeast Lipid Extract experience no irritation inherent to Retinol formulations.

### Example 10

Pollution Protectant Skin Cream with Yeast Lipid Extract
Ingredients in the composition

| Phase A | |
|---|---|
| Distilled Water | q.s. to 100% |
| Disodium EDTA | 0.02% |
| Allantoin, USP | 0.50% |
| Propylene Glycol, USP | 15.00% |
| Phase B | |
| High Molecular Weight Dimethiconol | 4.00% |
| Cyclomethicone | 8.00% |
| Dimethicone Copolyol | 3.70% |
| Caprilic Capric Triglycerides | 2.00% |
| Neopentyl Glycol Diheptanoate | 3.00% |
| Isodecane | 4.00% |
| Vitamin E Acetate | 0.20% |
| Oat Oil Complex | 1.00% |
| Sipeneo P-600 | 3.00% |
| Phase C | |
| Phenoxyethanol | 0.50% |
| Colloidal Oatmeal | 1.00% |
| Bisabolol | 0.05% |
| Phase D | |
| Yeast Lipid Extract | 3.00% |

Processing: Add Phase A ingredients with moderate lightning mixing. Heat to 50° C. mix for 15 minutes and cool back down to 25 C. Add all Phase B ingredients with lightening mixing at slow speed. Emulsify by slowly adding Phase A to Phase B with high mixing speed (cold temperature water-in-oil emulsion) and continue mixing. Slow mixing speed to moderate and add Phase C ingredients one by one while continuing to mix at 25° C. for 10 minutes and stop.

### Example 8

Yeast Whole Cell and Yeast Lipid Extract Facial Cleanser

A Conditioning Facial Exfoliating Cleanser Containing Yeast Lipid Extract Composition.

Ingredients in the Composition

| Phase A | |
|---|---|
| Distilled Water | q.s. to 100% |
| Disodium EDTA | 0.04% |
| Xanthan Gum | 0.15% |
| Acrylates Copolymer | 0.50% |
| Sodium Chloride | 0.90% |
| Phase C | |
| Sodium Lauryl Sulfate (30%) | 6.00% |
| Disodium Lauryl Sulfosuccinate | 10.00% |
| Sodium Methyl Cocyl Taurate | 8.00 |
| Decyl Glucoside | 3.00% |
| Ethylene Glycol Monosterate | 2.00% |
| Propylene Glycol Monostearate | 1.00% |
| Yeast Lipid Extract | 0.75% |
| Phase D | |
| Yeast Whole Cell | 1.25% |
| Phase E | |
| Fragrance | 0.25% |
| Benzyl Alcohol | 0.05% |
| Phenoxyethanol | 0.30% |

Procedure: Slowly add Phase A Ingredients one by one with moderate lightning mixing speed. Begin to heat to 65° C. and add Phase B ingredients one by one with slow mixing for 20 minutes to avoid foaming. Cool to 35° C. and add Phase C with continued slow mixing. Cool to 25° C. add Phase C ingredients one by one and mix for 10 minutes. Stop.

### Example 9

SPF Sunscreen Cream with Yeast Lipid Extract Tocopherol Liposomes

A Topical Sun Protection Product Containing a Comprehensive Yeast Lipid Extract Liposome for Sustain Release Antioxidant Benefits

Ingredients in the composition

| Phase A | |
|---|---|
| Distilled Water | q.s to 100% |
| Citric Acid | 0.20% |
| Glycerin 99.7% USP | 4.30% |
| Magnesium Aluminum Silicate | 0.50% |
| Xanthan Gum | 0.10% |
| Phase B | |
| C 12-15 Alkyl Benzoate | 6.20% |
| Zinc Oxide | 7.00% |
| Titanium Dioxide | 8.00% |
| Glyceryl Isostearate | 2.50% |
| Isohexadecane | 2.00% |
| Cetearyl Alcohol | 2.60% |
| Glyceryl Monostearate | 3.50% |
| PEG 100 Stearate | 4.10% |
| Phase C | |
| Phenoxyethanol | 0.50% |
| Methylisothiazolinone | 0.05% |
| Yeast Lipid Extract | 1.50% |
| Tocopherol Liposomes (made with yeast lipid extract | 1.0% |
| liposome) | |
| Fragrance | 0.20% |

Processing: Add or sprinkle Phase A ingredients one by one with moderate lightning mixing. Heat to 80° C. and mix for 15 minutes. Add all Phase B ingredients with lightening mixing at slow speed and heat to 80° C. Emulsify by slowly adding Phase B to Phase A with moderate mixing speed (oil-in-water emulsion) and continue mixing. Continue mixing and begin cooling to 40° C. and add Phase C ingredient one by one while continuing to mix and cool to 25° C. for 10 minutes and stop.

### Examples 10

Yeast Whole Cell/Peat Facial Mask

A Topical Peat Mask Containing Yeast Whole Cell to Deliver a Comprehensive Phospholipid Composition.

The natural acidic and osmotic conditions of wetlands such as Peat bogs sustains bacterial and yeast growth. Peat from a bog located in Midlands, Tullamore, Co. Offaly Ireland is characteristic of the highest organic content above 98%. The following composition was made using Peat screened to remove fibrous material from the Dunville bog located at Folio 8863. Peat from the surface containing bacteria and yeast was used after removing fibrous material as a base for the composition. Peat was used as a base along with Whole Cell Yeast of D. hansenii to fortify the phospholipid content creating a Peat—Phospholipid Fortified Facial mask containing phosphatidylcholine, phosphatidylserine, phosphatidylinositol, diphosphatidylglycerol, phosphatidylglycerol, phosphatidylethanolamine and a glycolipid. The two ingredients were mixed with a paddle mixer for 1 hour until homogeneous. The Facials Mask is brushed onto skin for a period of 10-15 minutes and removed with lukewarm towel. The product provides superior emollient aspects to the composition to compliment exfoliation effects that promote skin anti-aging properties.

Ingredients in the composition

| | |
|---|---|
| Peat (processed to remove fibrous matter) | 95% |
| Whole Cell Yeast | 5% |

### Example 11

Peat Emollient Facial Mask with Yeast Lipid Extract

A Topical Mask Containing Yeast Lipid Extract to Deliver a Comprehensive Phospholipid Composition.

Peat from a bog located in Midlands, Tullamore, Co. Offaly Ireland is characteristic of the highest organic content above 98%. The following composition was made using Peat from the Dunville bog located at Folio 8863. Peat from the surface containing bacteria and yeast was used after removing fibrous material as a base for the composition. For example, Bacillus acidiola and Debaryomyces hansenii are known to exist in peat bogs. Peat was used as a base along with Yeast Lipid Extract of D. hansenii to fortify the phospholipid content creating a Peat—Phospholipid Emollient Facial mask containing phosphatidylcholine, phosphatidylserine, phosphatidylinositol, diphosphatidylglycerol, phosphatidylglycerol, phosphatidylethanolamine and a glycolipid. The two ingredients were mixed with a paddle mixer for 1 hour until homogeneous. The Facials Mask is brushed onto skin for a period of 10-15 minutes and removed with lukewarm towel. The product provides superior emollient aspects to the composition to compliment exfoliation effects that promote skin anti-aging properties.

Ingredients in the composition

| | |
|---|---|
| Peat (processed to remove fibrous matter) | 98% |
| Yeast Lipid Extract | 2% |

### Example 12

White Peat Thermal Facial Mask with Yeast Lipid Extract

A Thermal Heat Generating Topical Mask Containing Yeast Lipid Extract to Deliver a Comprehensive Phospholipid Composition.

Ingredients in the composition

| Phase A | |
|---|---|
| Distilled Water | q.s. to 100% |
| Glycerin 99.7% USP, NF | 2.00% |
| Magnesium Aluminum Silicate | 2.00% |
| Xanthan Gum | 0.25% |
| Propanediol | 6.00% |
| PEG-75 Lanolin | 1.00% |
| Glycolic Acid | 5.00% |
| Phase B | |
| Rosehip Seed Oil | 1.00% |
| Caprylic Capric Trigylcerides | 1.00% |
| PPG-3 Isosteryl Methyl Ether | 2.00% |
| PEG-6 Stearate | 2.00% |
| PEG 32 Stearate | 1.50% |
| White Peat Extract | 2.00% |
| Peat-Yeast Lipid Extract | 2.50% |
| Kaolin | 10.00% |
| Phenoxyethanol | 0.50% |
| Phase C | |
| Vanillyl Butyl Ether | 0.10% |
| Fragrance | 0.10% |
| Phase D | |
| Yeast Lipid Extract | 2.00% |

Processing: Add Phase A Distilled Water, Glycerin and Propanediol with moderate lightning mixing. Heat to 80° C. and sprinkle other Phase A ingredients with continued lightening mixing at moderate speed for 15 minutes. Add all Phase B ingredients, heat to 80° C. with lightening mixing at slow speed. Emulsify by adding Phase B to Phase A and continue lightening mixing at moderate speed. Begin to cool to 30° C. and add Phase C. Cool to 25° C. and stop.

The Facials Mask is brushed onto skin for a period of 10-15 minutes and removed with lukewarm towel. The product provides superior emollient aspects to the composition to compliment exfoliation effects that promote skin anti-aging properties.

### Example 13

Anti-Psoriatic Cream with Yeast Lipid Extract
Ingredients in composition

| Phase A | |
|---|---|
| Distilled Water | q.s. to 100% |
| Disodium EDTA | 0.02% |
| Sodium Metabisulfite | 0.04% |
| Citric Acid | 0.05% |
| Sodium Benzoate | 0.10% |
| Potassium Sorbate | 0.10% |
| Glycerin 99.7% USP, FCC | 5.00% |
| Phase B | |
| Glyceryl Cocoate | 6.00% |
| Hydrogenated Coconut Oil | 4.00% |
| Cetereath-25 | 1.75% |
| Glyceryl Stearate and PEG 100 Stearate | 2.00% |
| Caprilic Capric Trigycerides | 2.00% |
| Medical Grade Lanolin | 2.50% |
| Pumpkin Seed Oil | 2.00% |
| Phase C | |
| Matricaria Recutita Extract | 0.90% |
| Phenoxyethanol | 0.50% |
| Phase D | |
| Yeast Lipid Extract | 6.00% |

Processing: Add Phase A ingredients with moderate lightning mixing. Heat to 80° C. Add all Phase B ingredients, heat to 80° C. with lightening mixing at slow speed. Emulsify by adding Phase B to Phase A and continue lightening mixing at moderate speed. Begin to cool to 40° C. and add Phase C ingredient. Cool to 35° C. and add Phase D ingredients. Continue mixing to 25° C. and stop. Use cream sparingly in affected topical areas.

### Example 14

Leave-On Hair Care Compositions with Yeast Lipid Extract Minoxidil Liposomes

A Leave on Hair Composition to Stimulate Hair Growth and Healthy Shine

Ingredients in the composition

| Phase A | |
|---|---|
| Cyclopentasiloxane | 8.50% |
| Dimethicone Crosspolymer | 6.20% |
| Dimethiconol | 2.50% |
| Cyclomethicone D4 | 4.50% |
| Dimethicone 200 | 1.00% |
| C11-13 Isoparaffin | 1.00% |
| Isodecahexane | 1.00% |
| Yeast Lipid Extract | 2.0% |
| Minoxidil Liposomes | 1.00% |
| (Liposomes made from a lipid yeast | |
| extract which comprise minoxidil) | |
| Fragrance | 0.20% |
| Phase B | |
| Distilled Water | q.s. to 100% |
| Sodium Chloride | 1.00% |
| Propylene Glycol | 15.00% |

Processing: Add Phase A ingredients with moderate lightning mixing. Add all Phase B ingredients with lightening mixing at slow speed. Emulsify by slowly adding Phase A to Phase B with high mixing speed (cold temperature water-in-silicone emulsion) and continue mixing for 10 minutes and stop.

### Example 15

Non-Drying Antibacterial Hand Sanitizer with Yeast Lipid Extract
A less drying alcoholic hand sanitizer
Ingredients in the composition

| Distilled Water | q.s. to 100% |
|---|---|
| Propylene Glycol, USP, NF | 2.00% |
| Glycerin 99.7% USP, NF | 2.00% |
| Hydroxypropylcellulose | 0.50% |
| Phase B | |
| Carbopol Ultra-20 | 0.60% |
| Phase C | |
| Dehydrated Ethyl Alcohol 200 | 70.00% |
| Monolaurin | 0.40% |
| Glyceryl Lactate | 0.40% |
| Octyldodecanol | 0.50% |
| Yeast Lipid Extract | 1.00% |

Procedure: Add Phase A ingredients one by one with moderate lightning mixing. Mix for 30 minutes at high speed. Sprinkle Phase B ingredients slowly and continue mixing at high speed for 30 minutes. Add Phase C ingredients to separate vessel with moderate mixing. Add Phase C to Phase A+B with continued slow mixing for 15 minutes and stop.

### Example 16

Ophthalmic Drops with Yeast Lipid Extract
An ophthalmic product to relive dryness in the eye
Ingredients in the composition

| Phase A | |
|---|---|
| Distilled Water | q.s to 100% |
| Disodium EDTA | 0.02% |
| Sodium Metabisulfite, NF | 0.04% |
| Glycerin 99.7%, NF | 5.00% |
| Lutrol 127 | 0.50% |
| Sodium Benzoate, NF | 0.02% |
| Citric Acid, USP | 0.05% |
| Potassium Sorbate, NF | 0.10% |
| Phase B | |
| Glyceryl Cocoate | 2.00% |
| Ceteareth 25 | 1.50% |
| Phase C | |
| Yeast Lipid Extract | 0.40% |

Procedure: Add Phase A ingredients one by one with moderate lightning mixing. Heat to 60° C. and add Phase B ingredients. Cool to 35° C. and add Phase C. Cool to 25° C. and stop.

Apply a drop of ophthalmic for relief of dry eyes.

### Example 17

Oral Spray with Yeast Lipid Extract
Oral spray product for relied of dry mouth
Ingredients in the composition

| Phase A | |
|---|---|
| Distilled Water | q.s. to 100% |
| Propylene Glycol USP | 15.00% |
| Modified Potato Starch | 0.80% |
| Phase B | |
| Potassium Sorbate | 0.10% |
| Sodium Benzoate | 0.10% |
| Disodium Phosphate | 0.20% |
| Citric Acid | 0.15% |
| Potassium Acesulfate | 0.25% |
| Xylitol | 0.10% |
| Vitamin E TGPS | 0.50% |
| Phase C | |
| Yeast Lipid Extract | 0.25% |

Procedure: Add Phase A ingredients one by one with moderate lightning mixing. Mix for 30 minutes at high speed. Slowly add Phase B ingredients one by one and continue mixing at high speed for 30 minutes. Slow mixing and Add Phase C with continued slow mixing for 5 minutes and stop.

Product is spray into oral cavity to relieve conditions of dry mouth, for example, such as experienced by patients with Sjogren's Syndrome.

### Example 18

Nasal Spray with Yeast Lipid Extract
A Nasal Spray, for example, for use in treatments for
improving memory and other neurological benefits
Ingredients in the composition

| Deionized Water, USP | q.s to 100% |
|---|---|
| Edetate Disodium | 0.02% |
| Xanthan Gum | 0.10% |
| Propylene Glycol | 1.00% |
| Glycerin 99.97 YSP | 2.50% |
| Polyethylene Glycol | 1.00% |
| Povidone | 0.25% |
| Sodium Phosphate Dibasic | 0.15% |
| Sodium Phosphate Monobasic | 0.25% |
| Yeast Lipid Extract in Cyclodextrin | 0.30% |

Procedure: Add each ingredient one by one at room temperature with moderate mixing for 30 minutes

### Example 19

Vaginal Lubricant with Yeast Lipid Extract
A lubricant to relieve vaginal dryness and promote more comfortable
intercourse
Ingredients in the composition

| Phase A | |
|---|---|
| Distilled Water | q.s to 100% |
| Xanthan Gum | 0.50% |
| Sodium Hyaluronate | 0.05% |
| Glycerin 99.7% USP | 7.00% |
| Sorbitol 70% USP | 1.50% |
| Polyacrylic Acid | 1.30% |
| Sodium Polyacrylate | 0.80% |
| Citric Acid | 0.05% |
| Sodium Benzoate | 0.02% |
| Potassium Sorbate | 0.10% |
| PEG-6 | 5.00% |
| Yeast Lipid Extract | 0.75% |

Procedure: Add Distilled Water—begin moderate speed lightning mixing and heat to 42° C. Add remaining ingredient in sequence and mix for 30 minutes. Cool to 25° C. and Stop

### Example 20

Beverage with Yeast Lipid Extract
A beverage to promote faster recovery after exercise
Ingredient in composition

| | |
|---|---|
| Brewed Green Tea | 97.00% |
| Citric Acid | 0.70% |
| Sucralose | 0.30% |
| Yeast Lipid Extract | 2.00% |

Procedure: Add all ingredient in sequence and mix for 10 minutes

### Example 24

Omega Acid Supplement with Yeast Lipid Extract

A gelatin cap made of vegetable glycerin as a nutritional supplement delivering Essential fatty acids including Omega 3 and Omega 6 with comprehensive phospholipids

Ingredients in the composition

| | |
|---|---|
| Krill Oil EPA/DHA | 80.00% |
| Yeast Lipid Extract | 20.00% |

### Example 21

Vitamin D Nutritional Supplement with Yeast Lipid Extract

A gelatin cap made of vegetable glycerin as a nutritional supplement delivering Vitamin D with comprehensive phospholipids with improved absorption

Ingredients in the composition

| | |
|---|---|
| Vitamin D 3 in Safflower Oil (6500 iu) | 85.00% |
| Yeast Lipid Extract | 15.00% |

### Example 22

Composition for growth stimulant with liposomes having humic acid entrapped to help in root hair signaling with phospholipids and combine it with other microorganisms listed below.

| Water | q.s | |
|---|---|---|
| Yeast Lipid Extract | 5% | |
| AcidGlomus intraradices | 3000 | propagulos/L |
| Pseudomonas fluorescens | 1 × 10E8 | UFC/L |
| Pseudomonas sp | 1 × 10E8 | UFC/L |
| Azotobacter sp | 1 × 10E9 | UFC/L |
| Azospirillum brasilenses | 1 × 10E9 | UFC/L |
| Bacillus subtilis | 1 × 10E9 | UFC/L |

### Example 23 Composition to Fight Nematodes

A compositions comprising a yeast lipid extract dried powder, made by drying a yeast lipid extract, admixed with

| | |
|---|---|
| Paecilomyces lilacinus | 1 × 10E9 UFC/Kg |
| Bacillus popilliae | 1 × 10E10 UFC/Kg |
| Bacillus thuringensis | 1 × 10E10 UFC/Kg |

1. A method of making a lipid yeast extract comprising a) mixing supernatant from a centrifuged lysed yeast culture with methanol and chloroform; and b) separating, after standing, lipids from the methanol and chloroform portion, to form a lipid yeast extract.

2. (canceled)

3. A medical or cosmetic composition comprising,
a) liposomes comprising a lipid yeast extract of Debaryomyces hansenii, wherein the lipid yeast extract is made by a) mixing supernatant from a centrifuged lysed Debaryomyces hansenii culture with methanol and chloroform; and b) separating, after standing, lipids from the methanol and chloroform portion, to form the lipid yeast extract; and
b) a medical or cosmetic ingredient.

4. A medical or cosmetic composition comprising,
a) at least 0.1% w/w whole yeast organisms
b) liposomes comprising lipids of a lipid yeast extract of Debaryomyces hansenii, wherein the lipid yeast extract is made by a) mixing supernatant from a centrifuged lysed yeast culture with methanol and chloroform; and b) separating, after standing, lipids from the methanol and chloroform portion, to form the lipid yeast extract;
and a medical or cosmetic ingredient.

5. The composition of claim 3, wherein the medical or cosmetic ingredient comprises at least one of absorbents, abrasives, anticaking agents, antifoaming agents, antimicrobial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances, flavor oils, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, soaps, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, vitamin derivatives, and natural extracts.

6. The composition of claim 3, wherein the medical or cosmetic ingredient comprises a soap.

7. The composition of claim 4, wherein the medical or cosmetic ingredient comprises at least one of absorbents, abrasives, anticaking agents, antifoaming agents, antimicrobial agents, binders, biological additives, buffering agents, bulking agents, chemical additives, cosmetic or medical biocides, denaturants, cosmetic or medical astringents, drug astringents, external analgesics, film formers, humectants, opacifying agents, fragrances, flavor oils, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, soaps, sunscreens, sunblocks, ultraviolet light absorbers or scattering agents, sunless tanning agents, antioxidants and/or radical scavengers, chelating agents, sequestrants, anti-acne agents, anti-inflammatory agents, anti-androgens, depilation agents, desquamation agents/exfoliants, organic hydroxy acids, vitamins, vitamin derivatives, and natural extracts.

8. The composition of claim 4, wherein the medical or cosmetic ingredient comprises a soap.
